SEQUENCE LISTING (5 sequences)

SEQ ID NO: 1 - protein, 60 aa, Artificial Sequence
  MadL2
YPESDSAKDDGNQEKEKALLVKVQERSDDGDYDEYDNDETTHTPDPSAPTARPRIREHQA

SEQ ID NO: 2 - protein, 61 aa, Artificial Sequence
  MadL1
YPERDSAKDGNEEQERALPVNVQERGEVADADYDDYDEEGTTPTVDPTAQTARPRLQGNQS

SEQ ID NO: 3 - protein, 11 aa, Artificial Sequence
  Peptide B
DFEEIPEEYLQ

SEQ ID NO: 4 - protein, 15 aa, Artificial Sequence
  Peptide B
DEDYAAIEASLSETF

SEQ ID NO: 5 - protein, 16 aa, Artificial Sequence
  Peptide B
PFDFEAIPEEYLDDES

(19) United States
(12) Patent Application Publication
(10) Pub. No.: US 2023/0416341 A1
(43) Pub. Date: Dec. 28, 2023

(54) IMPROVED THROMBIN INHIBITORS FOR TREATMENT OF THROMBOEMBOLIC CONDITIONS

(71) Applicants: THE UNIVERSITY OF SYDNEY, New South Wales (AU); THE HEART RESEARCH INSTITUTE LTD, New South Wales (AU)

(72) Inventors: Richard J PAYNE, New South Wales (AU); Emma WATSON, New South Wales (AU); Shaun Phillip JACKSON, New South Wales (AU)

(73) Assignees: THE UNIVERSITY OF SYDNEY, New South Wales (AU); THE HEART RESEARCH INSTITUTE LTD, New South Wales (AU)

(21) Appl. No.: 18/011,073

(22) PCT Filed: Jun. 19, 2020
(86) PCT No.: PCT/AU2020/050626

§ 371 Date: Dec. 16, 2022

(51) Int. Cl. C07K 14/81; A61P 7/02

(52) U.S. Cl. CPC C07K 14/811; A61K 38/00; C07K 2319/00; A61P 7/02

(57) ABSTRACT

The invention relates to the treatment of thromboembolic conditions, to fibrin clot formation and related thrombin activity, and to preparation of compounds, in particular, peptides and polypeptides for inhibiting, or for modifying the cleavage of fibrinogen by thrombin.

### FIELD OF THE INVENTION

The invention relates to the treatment of thromboembolic conditions, to fibrin clot formation and related thrombin activity, and to preparation of compounds, in particular, peptides and polypeptides for inhibiting, or for modifying the cleavage of fibrinogen by thrombin.

### BACKGROUND OF THE INVENTION

Reference to any prior art in the specification is not, and should not be taken as, an acknowledgment or any form of suggestion that this prior art forms part of the common general knowledge in Australia or any other jurisdiction.

Ischaemic stroke is caused by the development of a blood clot or embolus within the cerebral circulation and is the third most common cause of death globally. Stroke events are also the leading cause of disability worldwide and are associated with long, resource intensive and costly rehabilitation programs.

Currently the only approved pharmacological therapy for stroke to promote the rapid reperfusion of the ischaemic brain, thereby minimising a stroke event, is the intravenous (i.v.) delivery of the thrombolytic agent recombinant tissue plasminogen activator (rtPA). rtPA activates plasminogen to plasmin which subsequently degrades fibrin and other clot-associated proteins, thereby improving blood flow through the affected vessel.

Despite its widespread clinical use, rtPA-based therapy has a number of limitations in both efficacy and application. Of particular concern is that only 20-30% of patients will have complete artery re-canalisation following rtPA therapy and 20-30% of these patients will experience re-occlusion. This problem is believed to arise from clot-associated thrombin which retains activity for cleavage of fibrinogen to fibrin as rtPA derived plasmin degrades fibrin in the clot.

A further concern is the observation of increased incidence of intracranial haemorrhage (ICH) associated with rtPA therapy which effectively limits the dose of rtPA that can be given for thrombolytic therapy.

The significant limitations of rtPA therapy have sparked renewed interest in the development of improved thrombolytic therapies.

Thrombin plays a central role in clot formation, principally via the production of insoluble fibrin. As such, thrombin inhibitors have emerged as promising candidates for use as an adjunct therapy with rtPA.

To date, the indirect thrombin inhibitor heparin and the direct thrombin inhibitors (DTIs) hirudin and argatroban, have been investigated. Whilst an overall improvement in vessel re-canalisation was observed, the risk of bleeding and symptomatic ICH was increased with these co-therapies.

There is a need for thrombin inhibitors that can be used to improve ischaemic stroke outcomes.

There is also a need for thrombin inhibitors that can be used in conjunction with rtPA to improve ischaemic stroke outcomes.

There is also a need for thrombin inhibitors that can be used to improve other thrombogenic disease or coagulative disorders.

### SUMMARY OF THE INVENTION

The invention seeks to address one or more of the above mentioned needs or limitations and in one embodiment provides a peptide comprising, consisting essentially of, or consisting of an amino acid sequence shown in SEQ ID No: 1:

YPESDSAKDDGNQEKEKALLVKVQERSDDGDYDEYDNDETTHTPDPSAP
TARPRIREHQA.

In another embodiment, the invention provides a peptide comprising, consisting essentially of, or consisting of an amino acid sequence shown in SEQ ID No: 2:

YPERDSAKDGNEEQERALPVNVQERGEVADADYDDYDEEGTTPTVDPTA
QTARPRLQGNQS.

Preferably, the peptide consists of the amino acid sequence shown in SEQ ID No: 1 or SEQ ID No: 2. More preferably, the peptide consists of the amino acid sequence shown in SEQ ID No: 1.

In another embodiment, the peptide comprises, consists essentially of, or consists of a biologically active fragment or variant of an amino acid sequence shown in SEQ ID No:1 or SEQ ID No:2.

The peptide may be a recombinant, isolated or synthetic peptide.

In any aspect, the peptide at least partially inhibits thrombin.

Typically at least one residue of the peptide is a sulfated tyrosine or sulfated tyrosine mimic residue, preferably a sulfated tyrosine residue.

Preferably, the tyrosine at position 32 in SEQ ID No: 1 is sulfated and the tyrosine at position 35 is sulfated.

In another embodiment, the tyrosine at position 32 in SEQ ID No: 1 is sulfated. The tyrosine at position 35 in SEQ ID No: 1 may not be sulfated.

In another embodiment, the tyrosine at position 35 in SEQ ID No: 1 is sulfated. The tyrosine at position 32 in SEQ ID No: 1 may not be sulfated.

Preferably, the tyrosine at position 33 in SEQ ID No: 2 is sulfated and the tyrosine at position 36 is sulfated.

In another embodiment, the tyrosine at position 33 in SEQ ID No: 2 is sulfated. The tyrosine at position 36 in SEQ ID No: 2 may not be sulfated.

In another embodiment, the tyrosine at position 36 in SEQ ID No: 2 is sulfated. The tyrosine at position 33 in SEQ ID No: 2 may not be sulfated.

In another embodiment there is provided a fusion peptide of Formula 1:

A-B

wherein:
A is a peptide comprising, consisting essentially of, or consisting of an amino acid sequence shown in SEQ ID No: 1 or SEQ ID No: 2 and comprises one or more characteristics described above;
B is a peptide comprising, consisting essentially of, or consisting of an amino acid sequence shown in

SEQ ID No: 3:
DFEEIPEEYLQ
or
SEQ ID No: 4:
DEDYAAIEASLSETF
or
SEQ ID No: 5:
PFDFEAIPEEYLDDES.

Preferably, B binds to exosite I of thrombin.

Region A may be located N terminal to B. In another embodiment, B is located N terminal to A.

In one embodiment, A and B may be linked directly. In another embodiment, A and B may comprise a linker.

In any aspect, a peptide of the invention at least partially inhibits thrombin. Preferably, a peptide of the invention at least partially inhibits thrombin activity by at least 1%, 5%, 10%, 25%, 50%, 60%, 70%, 80% or 90% or more. Preferably, a peptide of the invention inhibits thrombin with an inhibition constant, Ki, of less than about 30 nM, less than about 20 nM, less than about 10 nM, less than about 5 nM, less than about 4 nM, less than about 3 nM, less than about 2 nM, less than about 1 nM, less than about 500 pM, less than about 200 pM.

In one embodiment, a peptide of the invention reduces clotting time by at least 20% compared with a known thrombin inhibitor, wherein the known thrombin inhibitor induces significant blood loss, on a molar equivalent basis of the peptide and the known thrombin inhibitor, as measured using an in vitro activated partial thromboplastin time (aPTT) assay described herein. Preferably, the known thrombin inhibitor is hirudin.

In one embodiment, a peptide of the invention comprises a clotting time of between about 60 s and about 200 s at concentrations of above about 4 μg/mL, as measured using an in vitro activated partial thromboplastin time (aPTT) assay.

In one embodiment, a peptide of the invention induces a clotting time of between about 60 s and about 200 s when administered to an individual in an amount of about 1 μg/kg to about 10 mg/kg of the individual, as measured ex vivo using an in vitro activated partial thromboplastin time (aPTT) assay.

In one embodiment, a peptide of the invention does not induce significant blood loss. Preferably, the peptide does not induce significant blood loss as measured using any assay which has the capacity to determine blood loss. More preferably, the peptide reduces blood loss by at least 10-fold compared with a known thrombin inhibitor, wherein the known thrombin inhibitor induces significant blood loss, on a molar equivalent basis of the peptide and the known thrombin inhibitor when administered to an individual. Preferably, the known thrombin inhibitor is hirudin or argatroban, more preferably hirudin.

In one embodiment, a peptide of the invention reduces symptomatic intracerebral haemorrhage (sICH) as compared with hirudin on a molar equivalent basis of the peptide and hirudin when administered to an individual.

In another embodiment there is provided a polynucleotide, preferably cDNA, comprising a nucleotide sequence encoding a peptide comprising, consisting essentially of, or consisting of an amino acid sequence of SEQ ID No: 1 or SEQ ID No: 2 as generally described above, or a peptide of Formula 1 described above, and vectors, expression constructs and cells containing said polynucleotide.

Preferably the cell contains a sulfotransferase enabling sulfation of tyrosine.

In one embodiment, a sulfotyrosine is genetically incorporated into a peptide according to the invention by amber codon suppression.

In another embodiment there is provided a pharmaceutical composition comprising a peptide comprising, consisting essentially of, or consisting of an amino acid sequence of SEQ ID No: 1 or SEQ ID No: 2 as generally described above, or a peptide of Formula 1 described above, and a pharmaceutically effective carrier, diluent or excipient.

The present invention also provides a method of preventing or treating a thromboembolic condition in an individual in need thereof, the method comprising, consisting essentially of or consisting of administering a therapeutically effective amount of a peptide of the invention to the individual, thereby preventing or treating the thromboembolic condition in the individual.

The present invention also provides a method of treating, or reducing the severity of a symptom of a thromboembolic condition in an individual, the method comprising, consisting essentially of or consisting of administering to the individual in need thereof a therapeutically effective amount of a peptide of the invention, thereby treating, or reducing the severity of the symptom of the thromboembolic condition in the individual.

In another aspect, the present invention also provides a method for inhibiting or minimising the progression of a symptom of a thromboembolic condition in an individual comprising, consisting essentially of or consisting of administering a therapeutically effective amount of a peptide of the invention to an individual who is experiencing a symptom of a thromboembolic condition, thereby inhibiting or minimising the progression of the symptom of the thromboembolic condition in the individual.

In a further aspect, the invention thus provides a method of reducing the severity of a thromboembolic condition in an individual in need thereof, comprising, consisting essentially of, or consisting of the steps of administering to the individual in need thereof a therapeutically effective amount of a peptide of the invention, thereby reducing the severity of the thromboembolic condition in the individual.

In another aspect, the present invention also provides a method for inhibiting or minimising the progression of clot formation associated with inflammation caused by a viral infection in an individual comprising, consisting essentially of or consisting of administering a therapeutically effective amount of a peptide of the invention to an individual who is experiencing inflammation caused by a viral infection, thereby inhibiting or minimising the progression of clot formation in the individual.

In a further aspect, the invention thus provides a method of reducing the severity of clot formation associated with inflammation caused by a viral infection in an individual in need thereof, comprising, consisting essentially of, or consisting of the steps of administering to the individual in need thereof a therapeutically effective amount of a peptide of the invention, thereby reducing the severity of clot formation in the individual.

In another aspect, the present invention also provides for the use of a peptide of the invention in the manufacture of a medicament for:
preventing or treating a thromboembolic condition in an individual;
treating or reducing the severity of a symptom of a thromboembolic condition in an individual;
inhibiting or minimising the progression of a symptom of a thromboembolic condition in an individual; and/or
reducing the severity of a thromboembolic condition in an individual;
inhibiting or minimising the progression of clot formation associated with inflammation caused by a viral infection in an individual; and/or
reducing the severity of clot formation associated with inflammation caused by a viral infection.

In another aspect, the present invention also provides a peptide of the invention for use in:
preventing or treating a thromboembolic condition in an individual;
treating or reducing the severity of a symptom of a thromboembolic condition in an individual;
inhibiting or minimising the progression of a symptom of a thromboembolic condition in an individual;
reducing the severity of a thromboembolic condition in an individual;
inhibiting or minimising the progression of clot formation associated with inflammation caused by a viral infection in an individual; and/or
reducing the severity of clot formation associated with inflammation caused by a viral infection.

In another aspect, the present invention also provides a pharmaceutical composition comprising, consisting essentially of, or consisting of a peptide of the invention, and a pharmaceutically acceptable carrier, diluent or excipient for use in:
preventing or treating a thromboembolic condition in an individual;
treating or reducing the severity of a symptom of a thromboembolic condition in an individual;
inhibiting or minimising the progression of a symptom of a thromboembolic condition in an individual;
reducing the severity of a thromboembolic condition in an individual;
inhibiting or minimising the progression of clot formation associated with inflammation caused by a viral infection in an individual; and/or
reducing the severity of clot formation associated with inflammation caused by a viral infection.

In another aspect, the invention provides a pharmaceutical composition for preventing or treating a thromboembolic condition in an individual, comprising as an active ingredient a peptide of the invention, and a pharmaceutically acceptable diluent, excipient or carrier. In one embodiment, the only active ingredient present in the composition is a peptide of the invention.

In another aspect, the invention provides a pharmaceutical composition for preventing or treating a thromboembolic condition in an individual, comprising as a main ingredient a peptide of the invention, and a pharmaceutically acceptable diluent, excipient or carrier. In one embodiment, the only active ingredient present in the composition is a peptide of the invention.

In any method, use or composition of the invention described above, the only active ingredient present in the composition is a peptide of the invention.

Alternatively, in any method, use or composition of the invention described above, the composition may optionally include a further active ingredient, preferably selected from: rtPA therapy, platelet inhibitor, or a combination thereof.

In any method, use or composition of the invention described above, the peptide of the invention may be administered in combination with one or more active substances. Preferably, the one or more active substances is selected from: rtPA therapy, platelet inhibitor, or a combination thereof. The administration of a peptide of the invention and active substance, may include simultaneous, sequential and/or separate (immediate or prolonged delays between) administration. Sequential and/or separate administration may be in any order. For example, the peptide of the invention may be administered prior to or after administration of the active substance, to the individual. Preferably, wherein the active substance is rtPA therapy, the peptide is administered after administration of the active substance.

In any method or use of the invention described herein, the peptide of the invention may be administered parenterally, preferably intravenously.

In another aspect, the present invention also provides a kit for use, or when used, in a method of the invention, the kit comprising, consisting essentially of, or consisting of:
a peptide of the invention; and optionally
written instructions describing the use of a peptide of the invention in a method of the invention.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE FIGURES

FIG. 1. Inhibition of human thrombin by differentially sulfated peptides of SEQ ID No:1 (MDL-2) homologues; Un (unsulfated), Y₃₂ (sulfated at Y₃₂), Y₃₅ (sulfated at Y₃₅), DS (sulfated at Y₃₂ and Y₃₅). (A) raw data; (B) Table of inhibition constants (Ki).

FIG. 2. Inhibition of human thrombin by differentially sulfated peptides of SEQ ID No:2 (MDL-1) homologues; Un (unsulfated), Y₃₃ (sulfated at Y₃₃), Y₃₆ (sulfated at Y₃₆), DS (sulfated at Y₃₃ and Y₃₆). (A) raw data; (B) Table of inhibition constants (Ki).

FIG. 3. Comparative anticoagulant and bleeding profiles of hirudin, argatroban and SEQ ID No:1 (MDL-2): (A) Activated partial thromboplastin time (aPTT) dose-response curves of mouse plasma comparing hirudin (n=5), argatroban (n=3) and MDL-2 (n=5); (B) MDL-2 clotting activity in vivo over time; (C) MDL-2 human plasma stability over time.

FIG. 4. Impact of hirudin, argatroban and SEQ ID No:1 (MDL-2) on mouse tail bleeding time.

FIG. 5. (A) Schematic of experimental thrombolysis—rtPA/anticoagulant combination and vessel recanalization. Carstair's histological staining was carried out on carotids excised from sham mice (non-injured vessels) (B), or those excised 10 minutes (C-D) or 1-hour post-carotid occlusion (E). Staining reveals platelets (blue/purple), fibrin (crimson/purple) and erythrocytes (yellow/orange) (5 μm cross-section, paraffin-embedded tissue). The collagen of the vessel wall is depicted in blue surrounding the thrombus.

FIG. 6. rtPA/anticoagulant combination and vessel recanalization: (A) no treatment; (B) rtPA (10 mg/kg); (C) rtPA (10 mg/kg) and hirudin (1 mg/kg); (D) rtPA (10 mg/kg) and SEQ ID No:1 (MDL-2) (10 mg/kg).

FIG. 7. Analytical HPLC trace (A) and mass spectrum (B) of purified SEQ ID No:1 (MadL2 (1-30) selenoester.

FIG. 8. Analytical HPLC trace (A) and mass spectrum (B) of purified SEQ ID No:1 (MadL2) DS (31-60) diselenide.

FIG. 9. Analytical HPLC trace (0 to 50% B over 5 min, 0.1% v/v TFA, λ=214 nm) and mass spectrum of (A) crude ligation, (B) crude deselenization, (C) crude neopentyl ester deprotection and (D) purified SEQ ID No:1 (MadL2) DS.

FIG. 10. MALDI-TOF mass spectrum of SEQ ID No:1 (MadL2) DS (A) 4000-20000 m/z and (B) 6600-7400 m/z.

FIG. 11. Analytical HPLC trace (A) and mass spectrum (B) of purified SEQ ID No:2 (MadL1) (1-28) selenoester.

FIG. 12. Analytical HPLC trace (A) and mass spectrum (B) of purified SEQ ID No:2 (MadL1) DS (29-61) diselenide.

FIG. 13. Analytical HPLC trace (0 to 50% B over 30 min, 0.1% v/v TFA, λ=214 nm) and mass spectrum of (A) crude ligation, (B) crude deselenization, (C) crude neopentyl ester deprotection and (D) purified SEQ ID No:2 (MadL1) DS.

FIG. 14. MALDI-TOF mass spectrum of SEQ ID No:2 (MadL1) DS (A) 4000-22000 m/z and (B) 6800-7300 m/z.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Disclosed herein are peptides that inhibit thrombin. The present inventors have surprisingly found that the thrombin inhibitor peptides disclosed herein do not induce substantial blood loss as compared to other known thrombin inhibitors, which makes them useful for preventing, treating, or delaying the progression of conditions associated with the production and/or action of thrombin. For example, the thrombin inhibitor peptides of the invention are useful for preventing, treating, or delaying the progression of thromboembolic conditions. In a particularly preferred embodiment, the thromboembolic condition is stroke. The thrombin inhibitor peptides of the invention may be useful for the treatment of stroke particularly when combined with rtPA therapy, optionally further combined with a platelet inhibitor.

It is proposed that these thrombin inhibitor peptides may be utilised to inhibit fibrin formation by clot-associated thrombin, thereby addressing some of the issues regarding re-canalisation associated with rtPA therapy. The thrombin inhibitor peptides may also be used to modify the binding affinity and/or function of other peptides that bind to and block function of the thrombin active site, and/or the thrombin exosites

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “and/or”, e.g., “X and/or Y” shall be understood to mean either “X and Y” or “X or Y” and shall be taken to provide explicit support for both meanings or for either meaning.

The use of the term “about” includes and describes the value or parameter per se. For example, “about x” includes and describes “x” per se. In some embodiments, the term “about” when used in association with a measurement, or used to modify a value, a unit, a constant, or a range of values, refers to variations of ±10%. For example, “about 60” in some embodiments includes 54-66.

### Selected Definitions

“Thrombin” is a serine protease having a central role in hemostasis through the conversion of fibrinogen to fibrin.

“Thrombin active site” is a catalytic site that cleaves a range of substrates including fibrinogen, fibrinopeptides, Factor V, Factor VIII, protease activated receptors (PARs), glycoprotein V, Factor XI, Factor XIII, ADAMTS13, protein C.

“Thrombin exosite II”, (also known as “heparin binding exosite”) is a positively charged recognition surface that influences substrate and cofactor binding, particularly fibrinogen, Gp1ba, and heparin. It may include residues R93, R101, R126, K236, K240, and R233.

“Thrombin exosite I” (also known as “fibrinogen binding exosite”) is a positively charged recognition surface that influences substrate and cofactor binding, particularly fibrinogen, Factor V, Factor VIII, ADAMTS13, Factor XIII, PAR-1, Factor XI and thrombomodulin. It may include residues K36, H71 R73, R75, Y76, and R77.

Thrombin, including the thrombin active site and exosites I and II are generally discussed in Lane D. et al. 2005 Blood J. 106:2605-2612.

“Sulfated tyrosine residue”, “tyrosine-O-sulfate”, “sTyr” and “TyrSO₃” is a residue arising from the transfer of a sulfur-containing group to the hydroxyl side chain of tyrosine. The residue may arise from the action of a tyrosylprotein sulfotransferase (TPST).

“Sulfated tyrosine mimic” refers to a mimic of a sulfated tyrosine residue, comprising but not limited to, tyrosine sulfonate and tyrosine difluorosulfonate.

The terms “thrombin inhibitor” and “anticoagulant” are used herein interchangeably.

The phrase “inhibits thrombin” is understood to mean that the peptide of the present invention inhibits or reduces thrombin activity. Thrombin activity refers to the ability of thrombin to convert fibrinogen to fibrin. Further, the activity is measured using a suitable in vitro, cellular or in vivo assay and the thrombin activity is blocked or reduced by at least 1%, 5%, 10%, 25%, 50%, 60%, 70%, 80% or 90% or more, compared to thrombin activity in the same assay under the same conditions but without the peptide.

“Clotting time” generally refers to the time required for formation of a fibrin clot. Typically clotting time is assessed by the activated partial thromboplastin time (aPTT) test.

“Blood loss” generally refers excessive bleeding or haemorrhage. Minor haemorrhage refers to clinically insignificant bleeding. Major haemorrhage refers to internal bleeding or when the individual loses sufficient amounts of blood to cause a haemodynamic instability. Blood loss may be measured clinically by measuring blood pressure, pulse rate, haemoglobin levels, urea levels in the blood, and/or by imaging. Indicators of major haemorrhage will be clear to a person skilled in the art. Blood loss may also be determined experimentally for example by measuring the total volume of blood collected from a mouse tail transection and may be quantified by measurement of haemoglobin (mg/dL) present in the total volume of blood lost during a specified observation period (for example 20 minutes). Total haemoglobin may be assessed via a commercially available Haemoglobin colorimetric assay kit (Cat #MAK115; Sigma-Aldrich).

As used herein, the phrase “significant blood loss” shall be understood to mean excessive blood loss and means an increase in the amount of haemoglobin by at least about 5-fold, 10-fold, 20-fold, 50-fold or more, compared to the amount of haemoglobin in the same assay under the same conditions but without the active. As used herein, the phrase “does not induce significant blood loss” shall be understood to mean an amount of haemoglobin no greater than about 5-fold, 4-fold-3-fold, 2-fold or less, than the amount of haemoglobin in the same assay under the same conditions but without the active.

As used herein the phase “reduces blood loss” may be understood to mean a decrease in the amount of haemoglobin by at least about 10-fold, 20-fold, 50-fold, 100-fold, 1000-fold or more, as compared to a known thrombin inhibitor, wherein the known thrombin inhibitor induces significant blood loss, on a molar equivalent basis of the peptide and the known thrombin inhibitor when administered to an individual.

Examples of “known thrombin inhibitors” which induce blood loss include: vitamin K antagonists (or coumarin anticoagulants), low molecular weight heparins (LMWHs), direct thrombin inhibitors (DTIs), or Factor Xa inhibitors. LMWHs include, for example, Bemiparin, Certoparin, Dalteparin, Enoxaparin, Nadroparin, Parnaparin, Reviparin, and Tinzaparin. DTIs include, for example, lepirudin, desirudin, bivalirudin, argatroban, dabigatran, and antithrombin Ill. Factor Xa inhibitors include, for example, apixaban, fondaparinux, rivaroxaban and edoxaban. In one example, the known thrombin inhibitor is hirudin. “Hirudin” is a low molecular weight peptide (7 kDa) comprised of 65 amino acids (Dodt et al., 1984 FEBS Lett., 165: 180-4) which prevents blood from coagulating by binding to exosite I and the thrombin active site (Stone and Hofsteenge, 1986 Biochem, 25:4622-28).

As used herein, the term “condition” refers to a disruption of or interference with normal function, and is not to be limited to any specific condition, and will include diseases or disorders.

As used herein, the terms “preventing”, “prevent” or “prevention” include administering a peptide of the invention to thereby stop or hinder the development of at least one symptom of a condition. This term also encompasses treatment of a subject in remission to prevent or hinder relapse.

As used herein, the terms “treating”, “treat” or “treatment” include administering a peptide described herein to thereby reduce or eliminate at least one symptom of a specified condition.

The phrase ‘therapeutically effective amount’ generally refers to an amount of one or more peptides of the invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Generally, a therapeutically effective amount is an amount effective to reduce thrombin generation or directly inhibit thrombin activity, sufficient to increase in vitro clotting times 2-3 fold or more depending on the therapeutic indication.

As used herein, the term “individual” shall be taken to mean any animal including humans, for example a mammal. Exemplary individuals include but are not limited to humans and non-human primates. For example, the individual is a human. Although the invention finds particular application in humans, the invention is also useful for therapeutic veterinary purposes. The invention is useful for domestic or farm animals such as cattle, sheep, horses and poultry; for companion animals such as cats and dogs; and for zoo animals.

A “peptide” as used herein refers to a polymer of amino acid residues and to variants and synthetic analogues of the same. Thus, this term applies to amino acid polymers in which one or more amino acid residues is a synthetic non-naturally-occurring amino acid, such as a chemical analogue of a corresponding naturally-occurring amino acid, as well as to naturally-occurring amino acid polymers. This term does not exclude modifications to either the side chain of an amino acid or the N- or C-terminus of the peptide.

The thrombin inhibitor peptides described herein may have conservative substitutions of at least one amino acid residue. Preferably, this conservative substitution does not alter the overall conformation or function of the peptide. Preferably the conservative substitution comprises a replacement of an amino acid with another having one or more similar properties.

Amino acids with similar properties are well known in the art. For example, polar/hydrophilic amino acids which may be interchangeable include asparagine, glutamine, serine, threonine, lysine, arginine, histidine, aspartate and glutamate; nonpolar/hydrophobic amino acids which may be interchangeable include glycine, alanine, valine, leucine, isoleucine, proline, tyrosine, phenylalanine, tryptophan and methionine; acidic amino acids which may be interchangeable include aspartate and glutamate and basic amino acids which may be interchangeable include histidine, lysine and arginine.

The peptides described herein may have non-, or unnatural amino acids incorporated. Unless otherwise specified, any amino acid may be natural or non-natural/unconventional. Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

“MDL-2”, “MadL-2”, and “madanin-like 2” as used herein refers to an amino acid sequence as shown in SEQ ID No:1.

“MDL-1”, “MadL-1”, and “madanin-like 1” as used herein refers to an amino acid sequence as shown in SEQ ID No:2.

Thrombin Inhibitor Peptides

In one embodiment there is provided a peptide comprising, consisting essentially of, or consisting of an amino acid sequence shown in SEQ ID No: 1:

YPESDSAKDDGNQEKEKALLVKVQERSDDGDYDEYDNDETTHTPDPSAP
TARPRIREHQA.

In another embodiment there is provided a peptide comprising, consisting essentially of, or consisting of an amino acid sequence shown in SEQ ID No: 2:

YPERDSAKDGNEEQERALPVNVQERGEVADADYDDYDEEGTTPTVDPTA
QTARPRLQGNQS.

Preferably, the peptide consists of the amino acid sequence shown in SEQ ID No: 1 or SEQ ID No: 2. More preferably, the peptide consists of the amino acid sequence shown in SEQ ID No: 1.

The peptide may comprise, consist essentially of, or consist of a biologically active fragment or variant of an amino acid sequence shown in SEQ ID No:1 or SEQ ID No:2.

As used herein, “biologically active fragment” describes a portion or sub-sequence of an amino acid sequence shown in SEQ ID No:1 or SEQ ID No:2, comprising a domain thereof, that has no less than 10%, preferably no less than 25%, more preferably no less than 50%, and even more preferably no less than 75%, 80%, 85%, 90%, or 95% of a biological activity of a peptide comprising an amino acid sequence shown in SEQ ID No:1 or SEQ ID No:2. Such activity may be evaluated using standard testing methods and bioassays recognizable by the skilled artisan in the field as generally being useful for identifying such activity.

A fragment of an amino acid sequence shown in SEQ ID No:1 or SEQ ID No:2 may constitute less than 60, less than 50, less than 40, less than 30, less than 20, or less than 10 contiguous amino acids of an amino acid sequence shown in SEQ ID No:1 or SEQ ID No:2. Multiple fragments of an amino acid sequence shown in SEQ ID No:1 or SEQ ID No:2 are also contemplated.

By “domain” (of a protein) is meant that part of a protein that shares common structural, physiochemical and functional features, for example hydrophobic, polar, globular, or helical domains, or properties, for example a protein binding domain such as an exosite II binding domain or thrombin active site domain, a receptor binding domain, a co-factor binding domain, and the like.

Also contemplated are variants of an amino acid sequence shown in SEQ ID No:1 or SEQ ID No:2 comprising one or more amino acid substitutions, insertions and/or deletions in SEQ ID No:1 (or a fragment thereof) or SEQ ID No:2 (or a fragment thereof), as compared to wild-type SEQ ID No:1/SEQ ID No:2.

Typically, and in relation to proteins, a “variant” protein has one or more amino acids that have been replaced by different amino acids. It is well understood in the art that some amino acids may be changed to others with broadly similar properties without changing the nature of the activity of the protein (i.e., conservative substitutions).

It will also be appreciated that one or more amino acid residues of a reference sequence, such as SEQ ID No:1/SEQ ID No:2 (or a fragment thereof), may be modified or deleted, or additional sequences added, without substantially altering the biological activity of an amino acid sequence shown in SEQ ID No:1/SEQ ID No:2 (or a fragment thereof). Such activity may be evaluated using standard testing methods and bioassays recognizable by the skilled artisan in the field as generally being useful for identifying such activity.

In one embodiment, a protein variant shares at least 80%, preferably at least 85% and more preferably at least 90%, 95%, 98%, or 99% sequence identity to a reference amino acid sequence set forth in SEQ ID No:1 or SEQ ID No:2.

Preferably, sequence identity is measured over at least 60%, more preferably over at least 75%, more preferably over at least 90% or more preferably over at least 95%, 98% or substantially the full length of the reference sequence.

In order to determine percent sequence identity, optimal alignment of amino acid and/or nucleotide sequences may be conducted by computerised implementations of algorithms (Geneworks program by Intelligenetics; GAP, BESTFIT, FASTA, and TFASTA in the Wisconsin Genetics Software Package Release 7.0, Genetics Computer Group, WI, USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389-402, 1997.

A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The peptide at least partially inhibits thrombin. The peptide may therefore remove or reduce clot-associated thrombin from a clot the subject of rtPA thrombolytic therapy, thereby minimising the incidence of reduced re-canalisation that is otherwise associated with rtPA therapy.

Typically at least one residue of the peptide is a sulfated tyrosine residue, or sulfated tyrosine mimic residue, preferably a sulfated tyrosine residue.

Preferably, the tyrosine at position 32 in SEQ ID No: 1 is sulfated and the tyrosine at position 35 is sulfated.

In another embodiment, the tyrosine at position 32 in SEQ ID No: 1 is sulfated. The tyrosine at position 35 in SEQ ID No: 1 may not be sulfated.

In another embodiment, the tyrosine at position 35 in SEQ ID No: 1 is sulfated. The tyrosine at position 32 in SEQ ID No: 1 may not be sulfated.

Preferably, the tyrosine at position 33 in SEQ ID No: 2 is sulfated and the tyrosine at position 36 is sulfated.

In another embodiment, the tyrosine at position 33 in SEQ ID No: 2 is sulfated. The tyrosine at position 36 in SEQ ID No: 2 may not be sulfated.

In another embodiment, the tyrosine at position 36 in SEQ ID No: 2 is sulfated. The tyrosine at position 33 in SEQ ID No: 2 may not be sulfated.

As described in the Examples herein, the sulfation of either or both tyrosine residues is associated with improvements in inhibition of thrombin activity.

The peptide may be a recombinant, isolated or synthetic peptide.

In one embodiment, sulfation of tyrosine residues may be achieved by expressing a nucleotide sequence encoding a peptide comprising, consisting essentially of, or consisting of an amino acid sequence of SEQ ID No: 1 or SEQ ID No: 2 in a cell that contains a tyrosylprotein sulfotransferase (TPST). In more detail, inorganic sulfate may be actived in the forms of adenosine-5′-phosphosulfate (APS) and 3′-phospho-adenosine-5′-phosphosulfate (PAPS) by ATP sulfurylase and APS kinase respectively. The activated sulfate may then be transferred to tyrosine by TPST in the Golgi body.

Another approach to obtain sulfated tyrosine residues in a peptide according to the invention is to utilise a recombinant expression system involving an amber codon suppression enabling sulfotyrosine to be incorporated into the peptide during the recombinant synthesis of the peptide (Liu, C C and Schultz P G, Nature Biotechnology 2006; Italia J S et al Nature Chemical Biology 2020).

In the Examples herein, the inventors provide a synthetic method enabling the production of homogenous compositions of sulfated peptides (i.e. compositions that contain a peptide having only one sulfation profile).

Thrombin Inhibitor Fusion Peptides

In further embodiments the invention relates to utilising the thrombin inhibitor peptides disclosed herein for design, modification and/or production of novel inhibitors of thrombin activity, in particular for inhibitors that prevent or at least minimise the cleavage of fibrinogen or fibrinopeptides by thrombin. These thrombin inhibitor peptides may provide for inhibitors that have an improved affinity for the binding to exosite I.

A thrombin inhibitor fusion peptide may be described according to Formula 1:

A-B

wherein:
A is a peptide comprising, consisting essentially of, or consisting of an amino acid sequence shown in SEQ ID No: 1 or SEQ ID No: 2 and has one or more characteristics described above;
B is a peptide comprising, consisting essentially of, or consisting of an amino acid sequence shown in

SEQ ID No: 3:
DFEEIPEEYLQ
or
SEQ ID No: 4:
DEDYAAIEASLSETF
or
SEQ ID No: 5:
PFDFEAIPEEYLDDES.

Preferably, B binds to exosite I of thrombin.

Region A may be located N terminal to B. In another embodiment, B is located N terminal to A.

In one embodiment, A and B may be linked directly.

In another embodiment, A and B may comprise a linker. The thrombin inhibitor fusion peptides of Formula 1 may comprise a linker in the form of a peptide sequence (for example a peptide comprising 2 or more amino acid residues such as Gly and Ala) or other polymer (for example, a diethylene glycol linker) that links B ( ) with A. Examples of peptide sequences comprise poly Ala or poly Gly peptides. The length of the linker peptides may be determined according to the molecular distance between different sites on thrombin.

Peptide Synthesis

The above described peptides and fusion peptides may be prepared by solid phase peptide synthesis. For example, a fusion peptide of Formula 1 may be prepared by a method comprising the steps of solid phase synthesis of A, solid phase synthesis of B, and ligation of A to B, or as an alternative in the final step, ligation of A to a linker, and ligation of the A-linker conjugate to B to from an A linker-B conjugate. Alternatively, A-B can be prepared by a single solid-phase peptide synthesis without ligation.

In other embodiments, the peptide may be synthesised by recombinant DNA technology. It is particularly preferred that the cell lines used in this technology are (i) capable of growing in the presence of inorganic sulfate and (ii) capable of assimilating inorganic sulfate into a biological system, in particular a system involving post translational modification of tyrosine residues. Such a cell line generally comprises a tyrosylprotein sulfotransferase in the Golgi body, enabling the formation of one or more tyrosine-O-sulfate residues. In certain embodiments the expression products may be heterogeneous with respect to tyrosine sulfation pattern. A homogenous population of tyrosine-sulfated isoforms can be obtained by purifying the expression products on a variety of separation systems including a chromatographic system enabling differentiation of isoforms on the basis of tyrosine-sulfated phenotype.

Assaying Activity of a Peptide

Binding to Thrombin

Peptides described herein may be investigated for thrombin selectivity by counter-screening against a panel of proteases comprising trypsin, chymotrypsin, elastase, papain, reptilase, and factor Xa and activated protein C from the blood coagulation cascade. Inhibitors are screened initially at a single concentration (5 μM) using a fluorescence polarisation assay as described above.

Molecular details of thrombin recognition and inhibition by the inhibitors described herein may be determined by solving the three-dimensional structures of their complexes with thrombin. Briefly, thrombin-inhibitor complexes are prepared in vitro and subjected to extensive sub-microlitre scale screenings for crystallisation conditions. Preliminary conditions are refined and optimised using custom grid screens. Determination of cryoprotection conditions and initial sample characterisation is performed using a X-ray diffractometer. High resolution X-ray diffraction data is collected at high brilliance synchrotron sources, ensuring an adequate level of detail in the resulting models. The structures are solved by molecular replacement techniques using the coordinates of unliganded human thrombin as search model and refined and interpreted using a computational platform. These data provide detail on the binding mode of the inhibitors and unveil key interactions with thrombin.

Determining Inhibitory Activity

Thrombin inhibitory activity of the peptides may be determined by measuring the inhibition of the amidolytic activity of human α-thrombin spectrophotometrically using Tos-Gly-Pro-Arg-p-nitroanilide as chromogenic substrate. Inhibition assays may be performed using 0.2 nM enzyme, 100 μM substrate, and increasing concentrations of peptide. The concentration of a peptide may be determined using a Direct Detect Infrared Spectrometer. The inhibition constant (Ki) of a peptide may be determined according to a tight-binding model by fitting the inhibited steady-state velocity data to the Morrison equation. Reaction progress may be monitored at 405 nm for 1-2 h on a microplate reader. Dose-response curves may be used to determine Ki values.

Preferably, a peptide of the invention at least partially inhibits thrombin activity by at least 1%, 5%, 10%, 25%, 50%, 60%, 70%, 80% or 90% or more.

Preferably, a peptide of the invention inhibits thrombin with an inhibition constant, Ki, of less than about 30 nM, less than about 20 nM, less than about 10 nM, less than about 5 nM, less than about 4 nM, less than about 3 nM, less than about 2 nM, less than about 1 nM, less than about 500 pM, less than about 200 pM. Preferably, a peptide comprising, consisting essentially of, or consisting of an amino acid sequence as shown in SEQ ID No:1 wherein at least one residue of the peptide is a sulfated tyrosine residue, inhibits thrombin with an inhibition constant, Ki, of less than about 3 nM. More preferably, a peptide comprising, consisting essentially of, or consisting of an amino acid sequence as shown in SEQ ID No:1 wherein the tyrosine at position 32 in SEQ ID No: 1 is sulfated and the tyrosine at position 35 is sulfated, inhibits thrombin with an inhibition constant, Ki, of less than about 200 pM, preferably about 160 pM. Preferably, a peptide comprising, consisting essentially of, or consisting of an amino acid sequence as shown in SEQ ID No:2 wherein at least one residue of the peptide is a sulfated tyrosine residue, inhibits thrombin with an inhibition constant, Ki, of less than about 20 nM. More preferably, a peptide comprising, consisting essentially of, or consisting of an amino acid sequence as shown in SEQ ID No:2 wherein the tyrosine at position 33 in SEQ ID No: 2 is sulfated, inhibits thrombin with an inhibition constant, Ki, of less than about 4 nM. More preferably, a peptide comprising, consisting essentially of, or consisting of an amino acid sequence as shown in SEQ ID No:2 wherein the tyrosine at position 33 in SEQ ID No: 2 is sulfated and the tyrosine at position 36 is sulfated, inhibits thrombin with an inhibition constant, Ki, of about 2 nM.

Determining Anticoagulant Activity

The anticoagulant activity (Thrombin Time, TT) of the peptides disclosed herein is determined by measuring their ability to prolong clotting of human plasma in vitro using a clinical TT assay. Briefly, human plasma from healthy donors (800 μL) is mixed with a concentration range of peptides, clotting initiated by addition of thrombin, and clotting time measured using a coagulometer. Peptides which prolong clotting time to ≥30 sec at a concentration of 50 nM may be further investigated in vitro/ex vivo for activated partial thromboplastin time (aPTT). In brief, pooled citrated plasma from C57BL6/J mice is pre-incubated with various concentrations (0-12 pg/mL) of peptides. aPTT of each plasma sample is quantified following addition of a coagulation activator and CaCl₂). In an ex vivo assay, mice are injected i.v. with peptides (fixed concentration determined from in vitro aPTT) and whole blood collected into sodium citrate (˜130 μL) 0, 15, 30, 45, 60, 90 and/or 120 min post-administration. aPTT is quantified on isolated plasma using a Siemans aPTT kit (Actin FS and CaCl₂) solutions), with fibrin generation monitored to measure clotting time.

The anticoagulant activity (aPTT) of the peptides disclosed herein may be determined by measuring their ability to prolong clotting of human plasma in vitro using the clinical aPTT assay (Siemens Healthineers, Erlangen, Germany). Human plasma (lyophilized powder from Stago, France) may be used to determine dose response of the peptides. Human plasma (100 μL) is mixed with a range of peptides (0 to 1000 μg/ml). APTT is quantified on the mixed plasma by BFT II Analyzer (Siemens), which uses a turbodensitometric detection technique.

Determining Clotting Time and Blood Loss

The inventors show in the Examples that peptides described above possess potent in vivo antithrombotic activity with reduced clotting time and reduced blood loss compared to known anticoagulants. This is advantageous because many anti-thrombotic drugs are limited in their use due to risk of bleeding (including intracranial bleeding in stroke therapy).

Blood loss is an important issue clinically, as bleeding risk is markedly increased in patients receiving anticoagulant therapy. Specifically, there is no effective treatment available against bleeding caused by known anticoagulants including hirudin. There is therefore a need for agents that reduce blood loss.

The inventors demonstrate the surprising effect that the sulfated polypeptides according to the present invention display significantly less bleeding compared to the known anticoagulant hirudin.

In one embodiment, a peptide of the invention reduces clotting time by at least 20% compared with a known thrombin inhibitor, wherein the known thrombin inhibitor induces significant blood loss, on a molar equivalent basis of the peptide and the known thrombin inhibitor, as measured using an in vitro activated partial thromboplastin time (aPTT) assay. Preferably, the clotting time is reduced by more than 20%, by more than 30%, by more than 40%, by more than 50%, or by more than 60% when compared to the known thrombin inhibitor. Preferably, the known thrombin inhibitor is hirudin or argatroban, more preferably hirudin.

In one embodiment, a peptide of the invention comprises a clotting time of between about 60 s and about 200 s at concentrations of above about 4 μg/mL, as measured using an in vitro activated partial thromboplastin time (aPTT) assay. Preferably, the clotting time is between about 60 s and about 150 s, more preferably between about 60 s and about 100 s.

In one embodiment, a peptide of the invention induces a clotting time of between about 60 s and about 200 s when administered to an individual in an amount of about 1 μg/kg to 10 mg/kg of the individual, as measured ex vivo using an in vitro activated partial thromboplastin time (aPTT) assay. Preferably, the clotting time is between about 60 s and about 150 s, more preferably between about 60 s and about 100 s. The amount of peptide may be at least about 1 μg/kg, 5 μg/kg, 10 μg/kg, 20 μg/kg, 50 μg/kg, 100 μg/kg, 250 μg/kg, 500 μg/kg, 750 μg/kg, 1 mg/kg, 2 mg/kg, 5 mg/kg, or 10 mg/kg,

In one embodiment, a peptide of the invention does not induce significant blood loss. Preferably, the peptide does not result in significant blood loss as measured using any assay described herein which has the capacity to determine blood loss. More preferably, the peptide reduces blood loss by at least 10-fold compared with a known thrombin inhibitor, wherein the known thrombin inhibitor induces significant blood loss, on a molar equivalent basis of the peptide and the known thrombin inhibitor when administered to an individual. In another embodiment, a peptide of the invention reduces blood loss by at least 20-fold, 50-fold, 100-fold, 1000-fold or more, compared with the known thrombin inhibitor. Preferably, the known thrombin inhibitor is hirudin or argatroban, more preferably hirudin.

Methods of Treatment

The disclosed peptides and compositions can be used for inhibiting thrombin activity, for example in anti-thrombotic amounts sufficient to inhibit thrombin activity in a subject, such as a human, in whom pathological thrombosis is not desired. The peptides of the invention are therefore useful for the prevention or treatment of a thromboembolic condition in mammals.

The present invention therefore provides a method of preventing or treating a thromboembolic condition in an individual in need thereof, the method comprising, consisting essentially of or consisting of administering a therapeutically effective amount of a peptide of the invention to the individual, thereby preventing or treating the thromboembolic condition in the individual.

The present invention also provides a method of treating, or reducing the severity of a symptom of a thromboembolic condition in an individual, the method comprising, consisting essentially of or consisting of administering to the individual in need thereof a therapeutically effective amount of a peptide of the invention, thereby treating, or reducing the severity of the symptom of the thromboembolic condition in the individual.

In another aspect, the present invention also provides a method for inhibiting or minimising the progression of a symptom of a thromboembolic condition in an individual comprising, consisting essentially of or consisting of administering a therapeutically effective amount of a peptide of the invention to an individual who is experiencing a symptom of a thromboembolic condition, thereby inhibiting or minimising the progression of the symptom of the thromboembolic condition in the individual.

In a further aspect, the invention thus provides a method of reducing the severity of a thromboembolic condition in an individual in need thereof, comprising, consisting essentially of, or consisting of the steps of administering to the individual in need thereof a therapeutically effective amount of a peptide of the invention, thereby reducing the severity of the thromboembolic condition in the individual.

The term “thromboembolic condition” as used herein includes myocardial infarction, stroke, pulmonary embolism, deep vein thrombosis, peripheral arterial occlusion, disseminated intravascular coagulation, cardiovascular and cerebrovascular thrombosis, thrombosis associated with post-operative trauma, obesity, pregnancy, side effects of oral contraceptives, prolonged immobilization, and hypercoaguable states associated with hematologic, immunologic or rheumatological disorders, unstable angina, arteriosclerosis, a reblockage of vessels after angioplasty with a balloon catheter, or blood clotting in hemodialysis, artificial heart valves, extracorporeal membrane oxygenation, or stent implantation.

The symptom of a thromboembolic condition may depend on the indication, and will be clear to the person skilled in the art. Examples of symptoms of stroke include: weakness or numbness or paralysis of the face, arm or leg on either or both sides of the body; impaired speech; dizziness; impaired balance; impaired vision; headache; acute delirium; cognitive impairment. Preferably, the individual is identified as having, or suspected of having, one or more symptoms of thromboembolic condition, preferably at least 2, 3 or 4 symptoms of thromboembolic condition.

In addition to its effects on the coagulation process, thrombin has been shown to activate a large number of cells (such as neutrophils, fibroblasts, endothelial cells, smooth muscle cells). Therefore, the peptides of the present invention may also be useful for the treatment or prophylaxis of adult respiratory distress syndrome, septic shock, septicemia, inflammatory responses which include, but are not limited to, edema, acute or chronic atherosclerosis, and reperfusion damage.

In another aspect, the present invention also provides a method for inhibiting or minimising the progression of clot formation associated with inflammation caused by a viral infection in an individual comprising, consisting essentially of or consisting of administering a therapeutically effective amount of a peptide of the invention to an individual who is experiencing inflammation caused by a viral infection, thereby inhibiting or minimising the progression of clot formation in the individual.

In a further aspect, the invention thus provides a method of reducing the severity of clot formation associated with inflammation caused by a viral infection in an individual in need thereof, comprising, consisting essentially of, or consisting of the steps of administering to the individual in need thereof a therapeutically effective amount of a peptide of the invention, thereby reducing the severity of clot formation in the individual.

The virus is preferably selected from: coronavirus, influenza, parainfluenza, respiratory syncytial virus (RSV), adenovirus, cytomegalovirus (CMV), Epstein-Barr virus (EBV), varicella zoster virus (VZV), dengue virus, rhinovirus, Herpes simplex virus and enteroviruses. More preferably, the virus is coronavirus or influenza. Even more preferably, the virus is severe acute respiratory syndrome coronavirus (SARS-CoV), Middle East respiratory syndrome coronavirus (MERS-CoV), or severe acute respiratory syndrome coronavirus 2 (SARS-CoV-2), most preferably SARS-CoV-2.

The peptides of the present invention may also be used as an anticoagulant in extracorporeal blood circuits, such as those necessary in dialysis and surgery (such as coronary artery bypass surgery).

The peptides of the present invention may be used as a monotherapy. In another embodiment, the peptides of the present invention may be administered in combination with: one or more thrombolytic agents, one or more platelet inhibitors, or a combination thereof. The invention provides for a dose of thrombolytic agent and/or platelet inhibitor when used in combination with the peptide that is at, or lower than the dose prescribed according to the approved indications.

As described above, at least 20-30% of patients who receive rtPA therapy for ischaemic stroke will have complete artery re-canalisation following rtPA therapy and of these, 20-30% will experience re-occlusion. Some consider that this arises when rtPA therapy reveals clot-entrapped thrombin which is understood to be bound to the clot via exosite II, enabling the active site of the thrombin to cleave fibrinogen and fibrinopeptides to amplify and build the clot. Thrombin inhibitors described herein may therefore be provided to an individual receiving rtPA therapy to enable the elution of clot-associated thrombin from a fibrin clot, thereby minimising the amount of thrombin at the clot which would otherwise cause fibrin production and clot expansion.

Thus, in one embodiment the peptides of the present invention may be used in combination with a thrombolytic agent, such as tissue plasminogen activator (natural or recombinant), streptokinase, urokinase, prourokinase, anisolated streptokinase plasminogen activator complex (ASPAC), animal salivary gland plasminogen activators, and the like. The peptides of the present invention may act in a synergistic fashion to prevent reocclusion following a successful thrombolytic therapy and/or reduce the time to reperfusion. The peptides of the present invention may also allow for reduced doses of the thrombolytic agent to be used and therefore minimize potential hemorrhagic side-effects. In a preferred embodiment, the thrombolytic agent is rtPA.

The peptides of the present invention may also be used in combination with a platelet inhibitor such as aspirin; triflusal; adenosine diphosphate receptor inhibitors including Cangrelor, Clopidogrel, Prasugrel, Ticagrelor, Ticlopidine; phosphodiesterase inhibitors such as Cilostazol; protease-activated receptor-1 antagonists such as Vorapaxar; glycoprotein IIB/IIA inhibitors such as Abciximab, Eptifibatide, Tirofiban; adenosine reuptake inhibitors such as Dipyridamole; and thromboxane inhibitors.

The peptides of the present invention may be used in combination with a thrombolytic agent, preferably tissue plasminogen activator, and a platelet inhibitor.

Compositions

Pharmaceutical compositions may be formulated for any appropriate route of administration, preferably parenteral administration. The term “parenteral” as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. Typically the thrombin inhibitor peptide is provided in the form of a composition adapted for i.v. administration.

Methods for preparing a peptide into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of a peptide of the invention dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a peptide of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

In one embodiment, the peptide is administered locally at the site where a thrombus has formed.

In another embodiment, the peptide is administered directly into the thrombus.

In another embodiment, the peptide is administered as a bolus. The bolus may be an intravenous bolus or a bolus plus infusion to maintain steady-state levels. Preferably, the peptide is administered according to the invention to the individual within 12 hours after the first identification of a thromboembolic condition, preferably within 3 hours, 2 hours, 1 hour, or less.

Administration preferably occurs by bolus injection or by intravenous infusion, preferably as soon as possible after the identification of a thromboembolic event.

The dosage administered depends on the age, health, and weight of the individual.

Typically the thrombin inhibitor peptide is provided in an amount of about 1 μg/kg to 10 mg/kg of the recipient.

### EXAMPLES

### Example 1—Peptide Synthesis

Peptides of SEQ ID No:1 and SEQ ID No:2 may be prepared by chemical synthesis, including diselenide selenoester ligation and deselenization, and solid phase peptide synthesis as described herein.

Materials

Peptide grade dimethylformamide (DMF) was obtained from Labscan. Amino acids, coupling reagents and resins for Fmoc-solid-phase peptide synthesis (SPPS) were obtained from either Novabiochem or GL Biochem. SPPS was performed in polypropylene syringes equipped with Teflon filters, purchased from Torviq. Analytical reversed-phase high-performance liquid chromatography (HPLC) was performed on either a Waters Acquity UPLC system equipped with PDA eλ detector (λ=210-400 nm), Sample Manager FAN and Quaternary Solvent Manager (H-class) modules or a Waters System 2695 separations module with a 2996 photodiode array detector. Peptides were analyzed using an XBridge BEH 5 μm, 2.1×150 mm wide-pore column (C-18) at a flow rate of 0.2 mL min⁻¹ on the HPLC system or Waters Acquity UPLC BEH 1.7 μm 2.1×50 mm column (C-18) at a flow rate of 0.6 mL min-1 on the UPLC system. Both instruments were run using a mobile phase composed of 0.1% trifluoroacetic acid in H₂O (Solvent A) and 0.1% trifluoroacetic acid in acetonitrile (Solvent B) in a linear gradient as indicated. The analysis of the chromatograms was conducted using Empower 3 Pro software (2010) and retention times (Rt min) of pure peptides and proteins are reported with the gradients specified.

Preparative and semi-preparative reversed-phase HPLC was performed using a Waters 600E Multisolvent Delivery System with a Rheodyne 7725i Injection valve (4 mL loading loop) and Waters 500 pump with a Waters 490E programmable wavelength detector operating at 214, 230, 254 or 280 nm. Preparative reversed-phase HPLC was performed using a Waters Sunfire C18 column (5 μm, 10×250 mm) at a flow rate of 7 mL min⁻¹. Semi-preparative reversed-phase HPLC was performed using a Waters XBridge-BEH300 wide-pore C18 column (5 μm, 10×250 mm) at a flow rate of 4 mL min⁻¹. Peptides and proteins bearing free sulfated tyrosine (sTyr) residues were purified using a mobile phase of 0.1% Formic Acid in water (Solvent A) and 0.1% Formic Acid in acetonitrile (Solvent B) using the indicated linear gradient. A mobile phase of 0.1% trifluoroacetic acid in water (Solvent A) and 0.1% trifluoroacetic acid in acetonitrile (Solvent B) was used in all other cases, using the linear gradients specified. After lyophilization, peptides were isolated as TFA or formate salts depending on the chromatographic eluent.

LC-MS was performed either on a Shimadzu LC-MS 2020 instrument consisting of a LC-M20A pump and a SPD-20A UV/Vis detector coupled to a Shimadzu 2020 mass spectrometer (ESI) operating in positive mode unless otherwise stated, or a Shimadzu UPLC-MS equipped with the same modules as the LC-MS system except for a SPD-M30A diode array detector. Separations were performed on the LC-MS system either on a Waters Sunfire 5 μm, 2.1×150 mm column (C-18), or wide-pore equivalent operating at a flow rate of 0.2 mL min⁻¹. Separations on the UPLC-MS system were performed using a Waters Acquity UPLC BEH 1.7 μm 2.1×50 mm column (C-8) at a flow rate of 0.6 mL min⁻¹. Separations were performed using a mobile phase of 0.1% formic acid in water (Solvent A) and 0.1% formic acid in acetonitrile (Solvent B) and a linear gradient of 0-50% B over 30 min on the LC-MS System and 0-50% B over 8 min on the UPLC-MS system.

Low-resolution mass spectra were recorded on a Shimadzu 2020 mass spectrometer (ESI) operating in positive or negative mode as indicated. Low Resolution MALDI-TOF mass spectra were recorded on a Bruker Autoflex™ Speed MALDI-TOF mass spectrometer operating in linear mode using a matrix of 10 mg/mL sinapinic acid in water/acetonitrile (1:1 v/v) with no TFA.

General Peptide Synthesis Procedures

Fmoc-Strategy SPPS General Procedures (100-200 μmol Scale)

2-Chlorotrityl Chloride Resin Loading

2-Chlorotrityl chloride resin (1.22 mmol/g loading) was swollen in dry CH₂Cl₂ for 30 min, followed by washing with CH₂Cl₂ (1 Ox 3 mL). A solution of Fmoc-Xaa-OH (0.7 mmol/g resin) and iPr₂NEt (4 eq. relative to resin functionalization) in CH₂Cl₂ (final concentration 0.125 M of amino acid) was added to the resin, which was shaken at room temperature for 16 h. The resin was then washed with CH₂Cl₂ (5×3 mL), DMF (5×3 mL) and CH₂Cl₂ (5×3 mL). The resin was then capped via treatment with 17:2:1 v/v/v CH₂Cl₂:MeOH:iPr₂NEt (5 mL) for 40 mins at room temperature. The resin was then washed again with CH₂Cl₂ (5×3 mL), DMF (5×3 mL) and CH₂Cl₂ (5×3 mL) prior to determination of the estimated loading of the first amino acid.

Rink Amide Resin Loading

Rink Amide resin (0.7 mmol/g loading) was swollen in dry CH₂Cl₂ for 30 min, followed by washing with CH₂Cl₂ (10×3 mL). The resin was treated with piperidine/DMF (1:4 v/v, 3 mL, 2×5 min) and then washed with DMF (5×3 mL), CH₂Cl₂ (5×3 mL) and DMF (5×3 mL). A solution of Fmoc-Xaa-OH (0.7 mmol/g resin), PyBOP (4 eq. relative to resin functionalization) and N-methyl-morpholine (8 eq. relative to resin functionalization) in DMF or NMP (final concentration 0.125 M of amino acid) was added to the resin, which was shaken at room temperature for 2 h. The resin was then washed with DMF (5×3 mL) and CH₂Cl₂ (5×3 mL) prior to capping via treatment with 9:1 v/v pyridine:Ac₂O (5 mL) for 5 mins at room temperature. The resin was then washed again with CH₂Cl₂ (5×3 mL), DMF (5×3 mL) and CH₂Cl₂ (5×3 mL) prior to determination of the estimated loading of the first amino acid.

Loading Estimation of the First Amino Acid

The resin was treated with piperidine/DMF (1:4 v/v, 3 mL, 2×5 min) and then washed with DMF (5×3 mL), CH₂Cl₂ (5×3 mL) and DMF (5×3 mL). The combined deprotection solutions were then made up to 10 mL with fresh piperidine/DMF (1:4 v/v). The solution was diluted 50-100 fold with fresh piperidine/DMF (1:4 v/v) and the UV absorbance of the piperidine-fulvene adduct measured (λ=301 nm, ε=7800 M⁻¹cm⁻¹) to estimate the amount of amino acid loaded onto the resin.

Peptide Assembly Via Iterative SPPS

Peptides were assembled by stepwise Fmoc-SPPS on a 0.05-0.2 mmol scale. Coupling of each amino acid (4 eq.) was achieved using PyBOP (4 eq.) and NMM (8 eq.) in DMF (0.1 M) over 1 h at room temperature. Capping steps were introduced after all steps unless otherwise specified using 0.3 M acetic anhydride and 0.3 M iPr₂NEt in DMF for 3 min at room temperature. Fmoc-deprotection steps were performed by treatment with 20% v/v piperidine in DMF at room temperature (2×5 min). Following each coupling, capping or deprotection step, the resin was washed with DMF (5×3 mL), CH₂Cl₂ (5×3 mL) and DMF (5×3 mL).

Coupling of Fmoc-Tyr[SO₃—CH₂C(CH₃)₃]—OH

A solution of amino acid (2.0 eq.), DIC (2.0 eq.) and HOAt (2.0 eq.) in DMF (0.1 M) was added to the resin and shaken at room temperature for 16 h. The resin was then washed with DMF (5×3 mL), CH₂Cl₂ (5×3 mL) and DMF (5×3 mL) and a capping step was performed as described above. Synthesis of the desired fragment was then completed using iterative Fmoc-SPPS.

Coupling of Boc-Asp(SePMB)-OH or Boc-Sec(PMB)-OH

A solution of amino acid (1.5 eq.), DIC (1.5 eq.) and HOAt (1.5 eq.) in DMF (0.05 M) was added to the resin and shaken at room temperature for 16 h. The resin was then washed with DMF (5×3 mL), CH₂Cl₂ (5×3 mL) and DMF (5×3 mL).

Peptide Assembly Via Iterative Fully Automated Microwave-Assisted SPPS

Peptides were assembled by stepwise microwave assisted Fmoc-SPPS on a Protein Technologies Symphony peptide synthesiser, operating on a 0.1-0.2 mmol scale. Activation of entering Fmoc-protected amino acids (0.3 M solution in DMF) was performed using 0.3 M Oxyma in DMF/0.3 M DIC in DMF (1:1:1 molar ratio), with a 4 equivalent excess over the initial resin loading. Coupling steps were performed for 45 mins at 25° C. Capping steps were introduced after each coupling step unless otherwise specified and performed by treatment with a 0.3 M acetic anhydride/0.3 M iPr₂NEt solution in DMF (1×3 min). Fmoc-deprotection steps were performed by treatment of the resin with a 20% v/v piperidine solution in DMF at room temperature (2×3 min). Following each coupling, capping or deprotection step, the resin was washed with DMF (4×30 sec). Upon complete assembly of the peptide chain, the resin was washed with CH₂Cl₂ (5×30 sec) and gently dried under nitrogen flow.

Cleavage from the Resin

Fully Deprotected Peptide

The resin-bound peptide was treated with an ice-cold TFA:iPr₃SiH:water mixture (90:5:5 v/v/v, 5 mL) and allowed to shake at room temperature for 2 h. At this point, the resin was filtered and washed with fresh cleavage cocktail. The combined cleavage solutions were worked-up as described below.

Side Chain Protected Peptide

The resin was washed with CH₂Cl₂ (5×3 mL) before treating with a solution of HFIP/CH₂Cl₂ (3:7 v/v, 4 mL) and shaken for 40 mins at room temperature. The resin was filtered and washed with CH₂Cl₂ (3×3 mL). The combined cleavage solutions and washes were concentrated under nitrogen flow, to afford the crude sidechain protected product.

Solution-Phase Selenoesterification

A solution of fully sidechain protected peptide in dry DMF (20 mM) was treated with diphenyl diselenide (10 eq.) and tributyl phosphine (10 eq.) at −30° C. for 4-5 h under an argon atmosphere. The solution was then concentrated under nitrogen flow and subject to the acidic deprotection conditions described for the fully deprotected peptide.

Work-Up and Purification

Ice-cold diethyl ether (30 mL) was added dropwise to the concentrated cleavage solution to precipitate the crude peptide. The precipitate was then collected via centrifugation and washed with further diethyl ether to remove any remaining scavengers. Residual diethyl ether was removed under gentle nitrogen flow and dissolved in 0.1% v/v TFA aqueous buffer (with minimal addition of MeCN to aid dissolution, if necessary). The crude peptide was analysed by LC-MS (ESI) and purified by RP-HPLC.

On Resin Boc Protection, Allyl Deprotection and Selenoesterification

After the synthesis of the sequence was completed, Fmoc deprotection was carried out as described above. The resin (10-90 μmol) was then treated with Boc Anhydride (2 eq., 4-40 mg) and pyridine (2 eq., 2-14.5 μL) in DMF (0.01-0.03 M) for 4 h at room temperature. The resin was then washed, as above, and treated with Pd(PPh₃)₄ (0.9 eq., 10-100 mg) and phenylsilane (40 eq., 50-450 μL) in DCM (0.01-0.03 M) for 1 h at room temperature. This procedure was then repeated once and the resin washed, as above. Finally, the resin was treated with DPDS (30 eq., 90-850 mg) and Bu₃P (30 eq., 75-675 μL) in DMF (0.01-0.03 M) for 3 h at 0° C. Once again, the resin was washed before being submitted to global cleavage conditions, as described above.

PMB Deprotection of Ct Fragments and Purification

The crude C-terminal fragments were dissolved (7 mg/mL) in a 12:5:3 v/v/v mixture of TFA:buffer (6 M Gn·HCl, 100 mM HEPES, pH 4-5):DMSO and allowed to stir at room temperature for 1-4 h before dilution in water prior to purification by RP-HPLC.

General Protocol for One Pot Ligation-Deselenization

A 5 mM solution of peptide diselenide dimer (1.0 eq.) in ligation buffer (6 M Gn·HCl, 100 mM Na₂HPO₄, adjusted to pH 6.3) was added to the appropriate peptide selenoester (2.0-3.0 eq.). The resulting solution was carefully readjusted to pH 6.2-7.5 with 1 M KOH and the reaction incubated at 25° C. for 20 min. Upon complete conversion to the corresponding ligation product (judged through UPLC-MS monitoring), a solution of TCEP (500 mM) and dithiothreitol (50 mM) in buffer (6 M Gn·HCl, 100 mM Na₂HPO₄, pH 4-5) was added to give a final 250 mM concentration of TCEP, a 25 mM concentration of dithiothreitol, and a 2.5 mM final concentration with respect to the peptide diselenide dimer starting material. The reaction was then incubated at 25° C. for 10 min. After UPLC-MS analysis showed completed conversion to the deselenized product the reaction mixture was incubated at 37° C. for 6 h to remove the neopentyl groups. The ligation mixture was then diluted and purified by RP-HPLC.

### Example 1A: Synthesis of SEQ ID No:1 Via Diselenide-Selenoester Ligation (DSL) and Deselenization

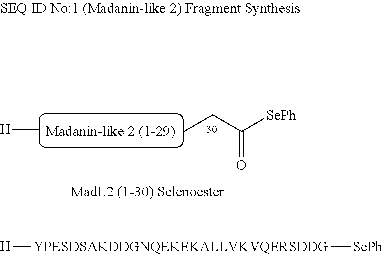

MadL2 (1-30) selenoester was prepared according to Fmoc-strategy SPPS, through a combination of automated and manual synthesis on 2-chlorotrityl chloride resin (100 μmol), with the N-terminal amino acid coupled as a Boc-protected amino acid. Upon completion of the resin-bound peptide fragment, HFIP-mediated cleavage and solution-phase selenoesterification with diphenyldiselenide (DPDS) (927 mg, 30 eq.) were completed as outlined in the general procedures. Removal of all acid labile protecting groups was achieved via treatment of the crude peptide selenoester with a solution of TFA/iPr₃SiH/H₂O (90:5:5 v/v/v, 4 mL) for 2 h at room temperature. The deprotection solution was then concentrated under nitrogen flow and the crude selenoester precipitated from ice-cold diethyl ether. Purification of the crude peptide by preparative RP-HPLC (0 to 30% B over 40 min, 0.1% v/v TFA) afforded MadL2 (1-30) selenoester as a fluffy white solid after lyophilization (27.88 mg, 7.98% yield).

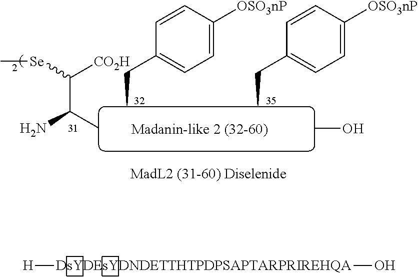

MadL2 DS (31-60) diselenide was prepared according to Fmoc-strategy SPPS, through a combination of automated and manual synthesis, on 2-chlorotrityl chloride resin (50 μmol). Removal of all of the acid labile protecting groups was achieved via treatment of the crude peptide with a solution of TFA/iPr₃SiH/H₂O (90:5:5 v/v/v, 4 mL) for 2 h at room temperature. The deprotection solution was then concentrated under nitrogen flow and the crude peptide from ice-cold diethyl ether. The PMB group was then removed as described above. Purification of the crude peptide by preparative RP-HPLC (0 to 50% B over 40 min, 0.1% v/v TFA) afforded MadL2 DS (31-60) diselenide as a fluffy white solid after lyophilization (4.36 mg, 2.24% yield).

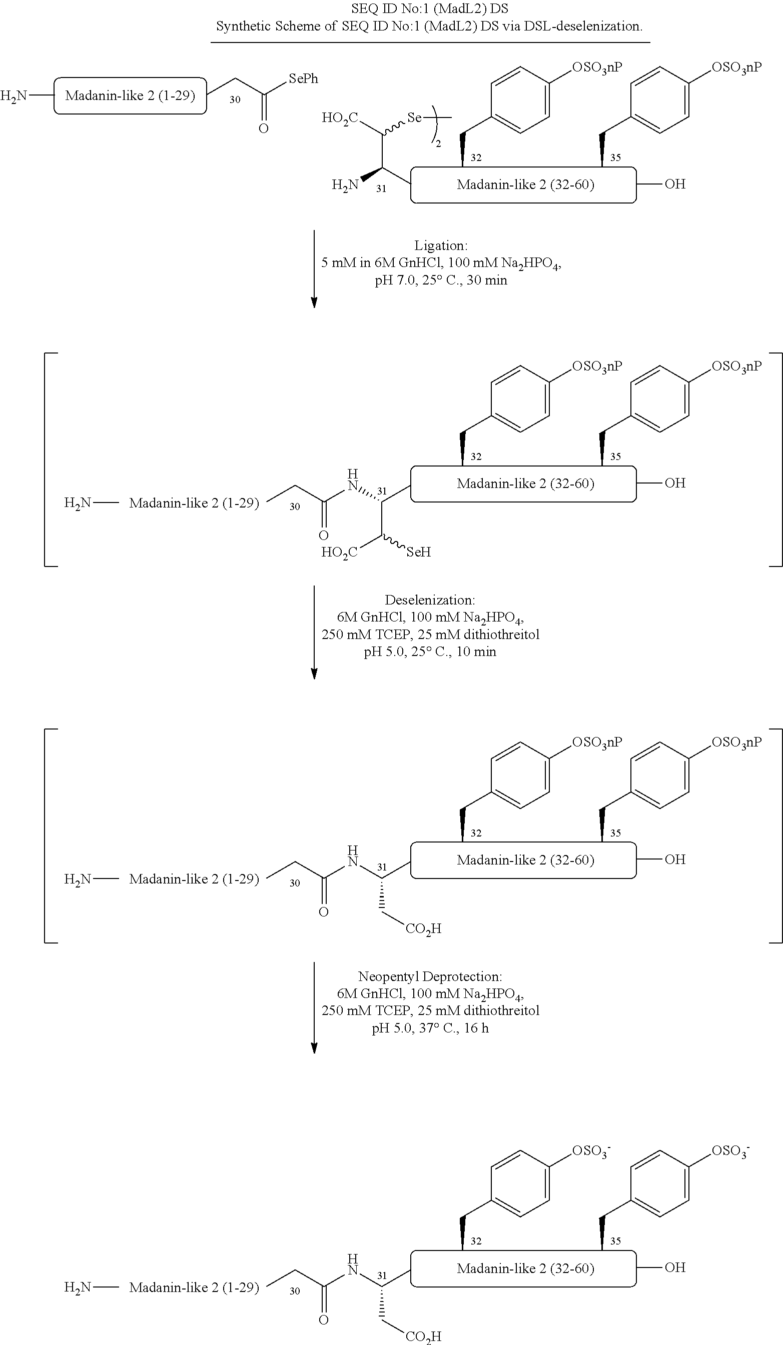

The one-pot peptide ligation of MadL2 (1-30) selenoester (2.7 mg, 0.77 μmol) and MadL2 DS (31-60) diselenide (2.0 mg, 0.52 μmol) followed by in situ deselenization was performed according to the general procedure, replacing 10% of the buffer volume with DMF. Purification via preparative HPLC (0 to 40% B over 40 min, 0.1% v/v Formic acid) followed by lyophilization afforded final protein MadL2 DS as a white solid (1.99 mg, 55%). Analytical HPLC (purified final product): Rt 3.84 min (0 to 50% B over 5 min, 0.1% v/v TFA, λ=214 nm); Calculated Mass [M+4H]⁴⁺: 1747.5 [M+5H]⁵⁺: 1398.2 [M+6H]⁶⁺: 1165.4, [M+7H]⁷⁺: 999.0, [M+8H]⁸⁺: 874.3, [M+9H]⁹⁺: 777.2 Mass Found (ESI) 1748.9 [M+4H]⁴⁺, 1399.2 [M+5H]⁵⁺, 1166.2 [M+6H]⁶⁺, 999.8 [M+7H]⁷⁺, 874.9 [M+8H]⁸⁺, 777.9 [M+9H]⁹⁺. High Res (ESI+): calcd for C₂₈₅H₄₃₇N₈₅O₁₁₇S₂: [M+8H]⁸⁺, 874.89037. Found: 874.88897 [M+8H]⁸⁺. Difference: 1.6 ppm.

### Example 1B: Synthesis of SEQ ID No:2 Via Diselenide-Selenoester Ligation (DSL) and Deselenization

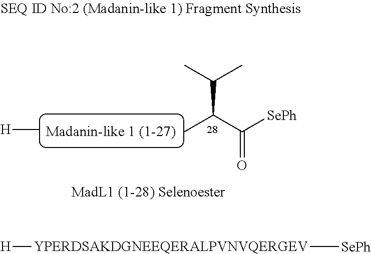

MadL1 (1-28) selenoester was prepared by loading of Fmoc-Val-OH to 2-chlorotritylchloride resin (62.5 μmol) and extended via Fmoc-strategy SPPS, through a combination of automated and manual synthesis, with the N-terminal amino acid coupled as a Boc-protected amino acid. Upon completion of the resin-bound peptide fragment, HFIP-mediated cleavage and solution-phase selenoesterification with diphenyldiselenide (DPDS) (590 mg, 30 eq.) were completed as outlined in the general procedures. Removal of all of the acid labile protecting groups was achieved via treatment of the crude peptide selenoester with a solution of TFA/iPr₃SiH/H₂O (90:5:5 v/v/v, 4 mL) for 2 h at room temperature. The deprotection solution was then concentrated under nitrogen flow and the crude selenoester precipitated from ice-cold diethyl ether. Purification of the crude peptide by preparative RP-HPLC (0% B for 5 min, 0% to 15% B over 5 min followed 15% to 35% B over 40 min, 0.1% v/v TFA) afforded MadL1 (1-28) selenoester as a fluffy white solid after lyophilization (56 mg, 27% yield).

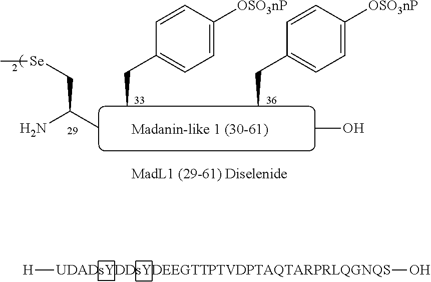

MadL1 DS (29-61) diselenide was prepared according to Fmoc-strategy SPPS, through a combination of automated and manual synthesis, on 2-chlorotrityl chloride resin (50 μmol). Removal of all of the acid labile protecting groups was achieved via treatment of the crude peptide with a solution of TFA/iPr₃SiH/H₂O (90:5:5 v/v/v, 4 mL) for 2 h at room temperature. The deprotection solution was then concentrated under nitrogen flow and the crude peptide from ice-cold diethyl ether. The PMB group was then removed as described above. Purification of the crude peptide by preparative RP-HPLC (0 to 35% B over 5 min, 35% B for 5 min followed by 35% to 55% B over 40 min, 0.1% v/v TFA) afforded MadL1 DS (29-61) diselenide as a fluffy white solid after lyophilization (6.2 mg, 3% yield).

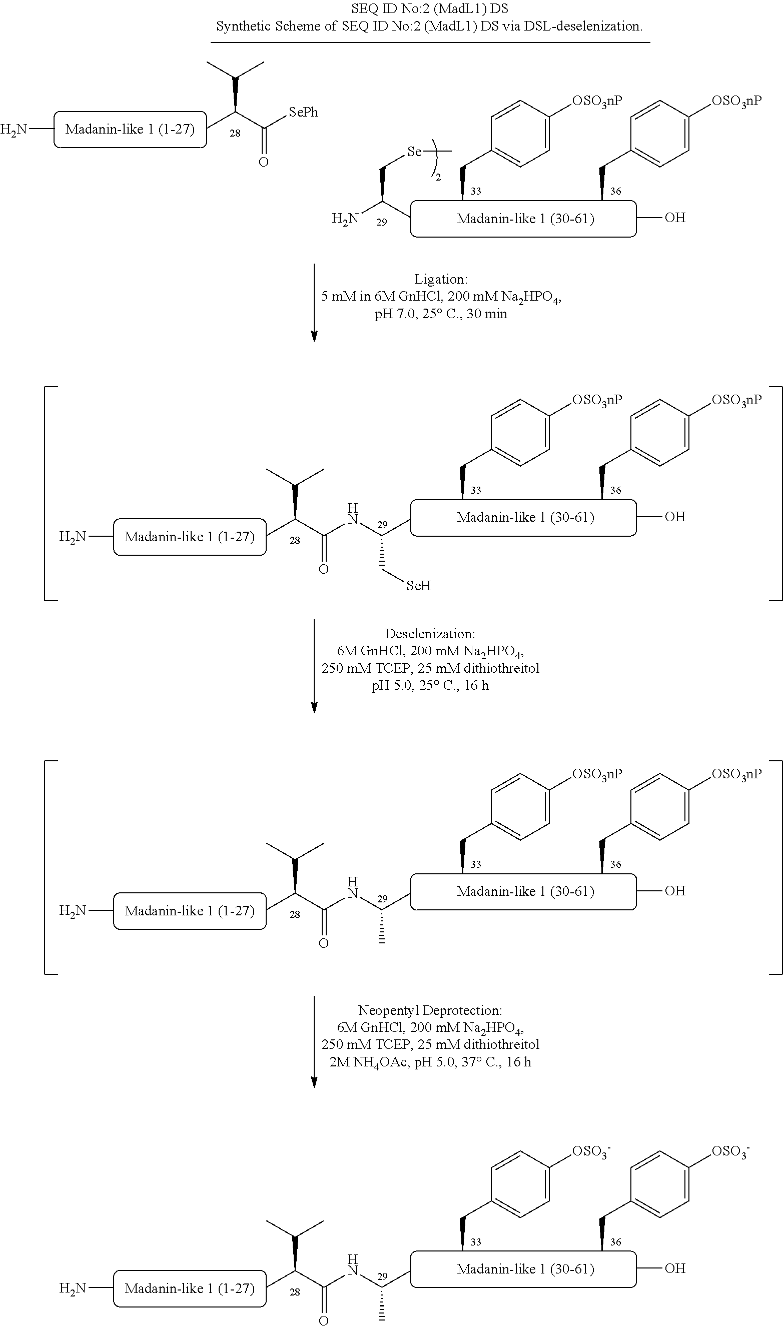

The one-pot peptide ligation of MadL1 (1-28) selenoester (2.5 mg, 0.75 μmol) and MadL1 DS (29-61) diselenide (2.0 mg, 0.50 μmol) followed by in situ deselenization was performed according to the general procedure. Purification via preparative HPLC (0% for 5 min, 0% to 40% B over 40 min, 0.1% v/v Formic acid) followed by lyophilization afforded final protein MadL1 DS as a white solid (2.1 mg, 67%). Analytical HPLC (purified final product): Rt 26.0 min (0 to 50% B over 30 min, 0.1% v/v TFA, λ=214 nm); Calculated Mass [M+4H]⁴⁺: 1740.0 [M+5H]⁵⁺: 1392.2 [M+6H]⁶⁺: 1160.4 Mass Found (ESI) 1740.3 [M+4H]⁴⁺, 1392.4 [M+5H]⁵⁺, 1160.5 [M+6H]⁶⁺.

### Example 1C: Synthesis of SEQ ID No:1 Via Solid Phase Peptide Synthesis

Synthesis of SEQ ID No: 1 (MDL2) (1-60) Y32, Y35 on single bead via SPPS
YPESDSAKDDGNQEKEKALLVKVQERSDDGD [symbol] DE [symbol] DNDETTHTPDPSAPTA
RPRIREHQA

MadL2 (1-60) Y32, Y35 was from (100 μmol) of preloaded Fmoc-Ala-Wang polystyrene resin via Fmoc-SPPS at 50° C. on a SYRO peptide synthesiser using the protocol described below, incorporating neopentyl-protected tyrosine sulfates at positions Y32 and Y35 (indicated as sY) using the commercially available Fmoc-Tyr[SO₃CH₂C(CH₃)₃]—OH building block as described in the general procedure above. Fmoc-Asp(OtBu)-(Dmb)Gly-OH dipeptides were also incorporated as required (indicated DG) to prevent aspartimide formation, using the same synthetic conditions. Removal of all acid labile protecting groups with concomitant cleavage from the resin support was achieved via treatment with a solution of TFA/iPr₃SiH/H₂O (90:5:5 v/v/v, 4 mL) for 2 h at room temperature. The deprotection solution was then concentrated under nitrogen flow and the crude peptide precipitated from ice-cold diethyl ether. Purification of the crude peptide by preparative RP-HPLC (0 to 40% B over 40 min, 0.1% v/v TFA) afforded MadL2 (1-60) with neopentyl-protected sTyr residues at Y32, Y35 as a fluffy white solid following lyophilisation. The neopentyl protected peptide was then dissolved to a concentration of 2.5 mM in 6M Gn·HCl, 0.1 M Na₂HPO₄, 250 mM TCEP, 25 mM DTT, pH 5 and incubated at 37° C. for 16 h to remove the neopentyl protecting groups, followed by preparative RP-HPLC (0 to 40% B over 40 min, 0.1% v/v formic acid) to afford MadL2 (1-60) Y32, Y35 as a fluffy white solid following lyophilisation. Yield: 40.6 mg, 5.7%. Note: all characterisation data was consistent with that obtained for material derived from the ligation strategy.

Biotage SYRO I Automated Peptide Synthesizer Protocol

Fmoc deprotection: The resin was treated with piperidine:DMF (1.6 mL of 20% for 2×4 min). The resin was filtered and washed with DMF (4×1.7 mL).

Coupling of proteinogenic amino acids: Fmoc-AA-OH (0.8 mL, 0.5 M), DIC (0.8 mL, 0.5 M), and Oxyma (0.8 mL, 0.55 M) were dispensed from stock solutions in DMF onto the resin. The reaction was heated to 50° C. and mixed for 30 min. The resin was filtered and washed with DMF (4×2.5 mL).

Capping: The resin was treated with a capping solution containing 5% acetic anhydride and 10% iPr2NEt in DMF (0.8 mL) for 6 min. The resin was filtered and washed with DMF (4×2.5 mL).

### Example 2—Biological Data

Inhibition of Thrombin by Sulfated Forms of SEQ ID No:1 and SEQ ID No:2

The inhibition of the amidolytic activity of human α-thrombin (Haematologic Technologies) was followed spectrophotometrically using Tos-Gly-Pro-Arg-p-nitroanilide (Chromozym TH; Roche) as chromogenic substrate. Inhibition assays were performed using 0.2 nM enzyme, 100 μM substrate, and increasing concentrations of peptide. The concentration of each peptide was determined using a Direct Detect Infrared Spectrometer (Millipore). The inhibition constants (Ki) of each peptide was determined according to a tight-binding model by fitting the inhibited steady-state velocity data to the Morrison equation (Williams J W & Morrison J F (1979)). All reactions were carried out at 37° C. in 50 mM Tris-HCl pH 8.0, 50 mM NaCl, 1 mg/mL BSA in 96-well microtiter plates. Reaction progress was monitored at 405 nm for 1-2 h on a Synergy2 Multimode microplate reader (BioTek). Dose-response curves were used to determine the Ki values using Prism 8.0 (GraphPad Software). For each peptide, at least two independent experiments with duplicate reactions were performed, together with control reactions in the absence of enzyme. For all curves, the goodness of fitting parameter R2 was between 0.989 and 0.999.

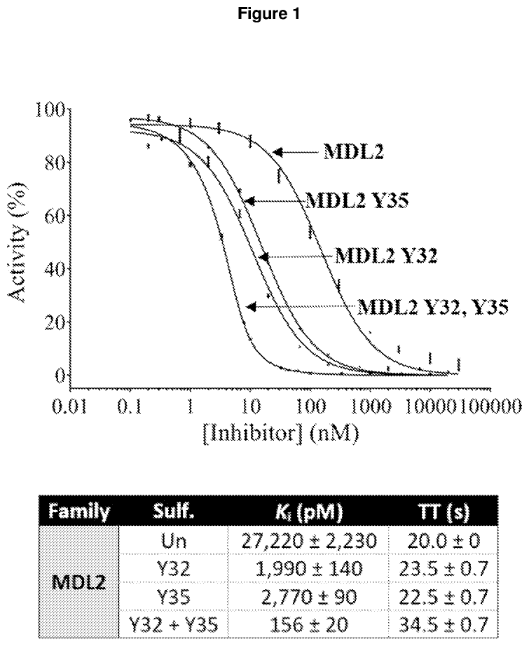

The inventors demonstrate, as shown in FIG. 1, that sulfated peptides of SEQ ID No:1 inhibit thrombin with a Ki of less than about 3 nM. Further, the doubly sulfated variant of this peptide is over two orders of magnitude more potent as inhibitors of human thrombin in vitro than the unsulfated counterpart.

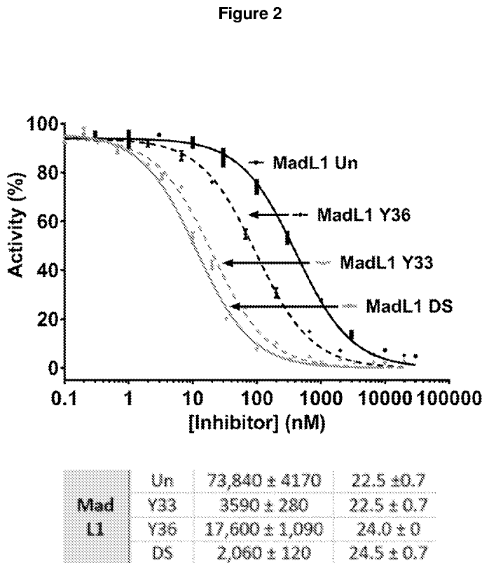

The inventors demonstrate, as shown in FIG. 2, that sulfated peptides of SEQ ID No:2 inhibit thrombin with a Ki of less than about 20 nM. Further, the doubly sulfated variant of this peptide is an order of magnitude more potent as inhibitors of human thrombin in vitro than the unsulfated counterpart.

Clotting and Bleeding Time of a Peptide of SEQ ID No:1

Activated Partial Thromboplastin Time (aPPT) Dose-Response Curve of Mouse Plasma for SEQ ID No:1 Compared with Hirudin and Argartroban.

The anticoagulant activity (Thrombin Time, TT) of the peptides disclosed herein is determined by measuring their ability to prolong clotting of human plasma in vitro using a clinical TT assay. Briefly, human plasma from healthy donors (800 μL) is mixed with a concentration range of peptides, clotting initiated by addition of thrombin, and clotting time measured using a coagulometer. Peptides which prolong clotting time to ≥30 sec at a concentration of 50 nM may be further investigated in vitro/ex vivo for activated partial thromboplastin time (aPTT). In brief, pooled citrated plasma from C57BL6/J mice is pre-incubated with various concentrations (0-12 μg/mL) of peptides. aPTT of each plasma sample is quantified following addition of a coagulation activator and CaCl₂). In an ex vivo assay, mice are injected i.v. with peptides (fixed concentration determined from in vitro aPTT) and whole blood collected into sodium citrate (˜130 μL) 0, 15, 30, 45, 60, 90 and/or 120 min post-administration. aPTT is quantified on isolated plasma using a Siemans aPTT kit (Actin FS and CaCl₂) solutions), with fibrin generation monitored to measure clotting time.

The anticoagulant activity (aPTT) of the peptides disclosed herein may be determined by measuring their ability to prolong clotting of human plasma in vitro using the clinical aPTT assay (Siemens Healthineers, Erlangen, Germany). Human plasma (lyophilized powder from Stago, France) may be used to determine dose response of the peptides. Human plasma (100 μL) is mixed with a range of peptides (0 to 1000 μg/ml). APTT is quantified on the mixed plasma by BFT II Analyzer (Siemens), which uses a turbodensitometric detection technique.

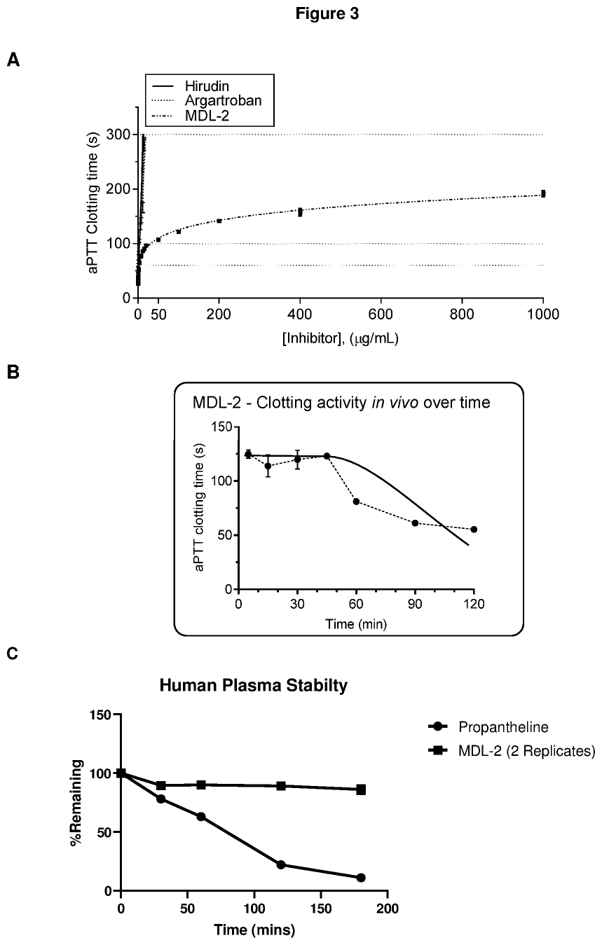

The inventors investigated the ability of a peptide of SEQ ID No:1 to prolong clotting time in an in vitro activated partial thromboplastin time (APTT) assay (FIG. 3A). These studies have demonstrated that while hirudin and argartroban exhibited an AAPT of greater than 200 s at concentrations above about 12 μg/mL, SEQ ID No:1 remained within a safer clotting time range with an AAPT of between about 60 s to about 200 s at concentrations of between about 4 μg/mL to about 600 μg/mL.

SEQ ID No:1 (MDL-2) Clotting Activity In Vivo Over Time

Mice were administered MDL-2 (10 mg/kg), and blood collected via submandibular (cheek) bleed over the indicated time course. For each sample, plasma was isolated and aPTT quantified in vitro using an activated partial thromboplastin time (aPTT) assay as describe herein.

Plasma Stability

Plasma stability of a peptide of SEQ ID No:1 (MDL-2) was determined using a modified method previously described by Teufel et al. (Teufel, Bennett et al. 2018). Positive control: Propantheline bromide (10 mM stock in DMSO).

MDL-2 (5 mM stock in 0.05M aq. NaOH) and propantheline bromide were added to human plasma (Sigma Aldrich citrated pooled plasma) to a final concentration of 200 μM. MDL-2 and the positive control were incubated at 37° C. for 0, 30, 60, 120 or 180 minutes before being quenched with three volumes of 1:1 v/v MeOH:MeCN. The samples were centrifuged at 13,500 rpm for 5 minutes before removing an aliquot of the supernatant (50 μL) and analysed by reverse-phase UHPLC and mass spectrometry. The area under the starting peak (normalised to total area under the chromatogram) was used to quantify the amount of uncleaved peptide remaining.

As shown in FIG. 3C, a peptide of SEQ ID No:1 (MDL-2) remains stable in plasma for up to about 180 mins.

Blood Loss

Blood loss was assessed by carrying out a 3 mm tail transection (lop) in rodents, to assess blood loss following compound/drug administration. Tail lop was performed using a standard transection 3 mm from the tip of the tail (Schoenwaelder et al, 2011, Blood). The tail was immediately immersed in pre-warmed sterile saline (37° C.) and time to bleeding cessation and rebleeding events monitored for 30 minutes following transection. Blood samples were centrifuged at 4,000 rpm for 15 minutes, and the pellet resuspended in 2 ml of RBC lysis buffer for at least 30 minutes at room temperature. Blood loss was quantified using a Haemoglobin assay kit, as per the manufacturer's instructions (Cat #MAK115; Sigma-Aldrich), using a haemoglaobin standard, and measurements taken using the CLARIOstar plate-reader.

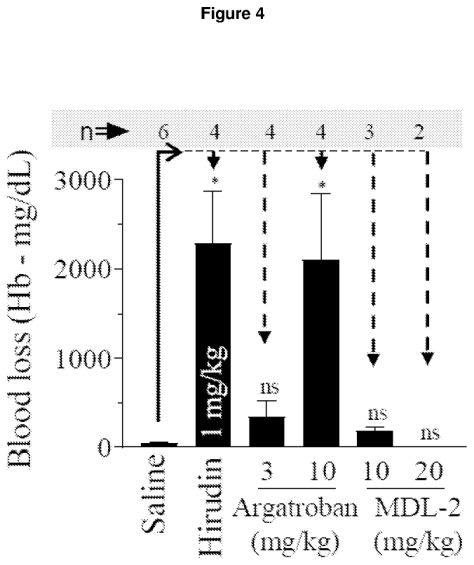

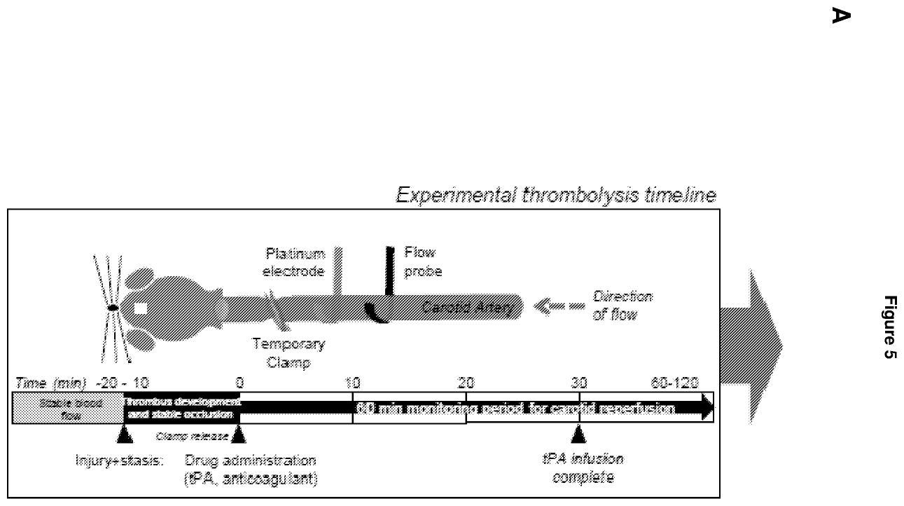

As shown in FIG. 4, a peptide of SEQ ID No:1 (MDL-2) results in significantly reduced blood loss at concentrations of 10 mg/kg or 20 mg/kg compared with hirudin at 1 mg/kg (over about 2000 mg/dL), and argatroban at 10 mg/kg (over about 2000 mg/dL).

Model of Experimental Thrombosis and Thrombolysis in Rodents

Thrombosis: Electrolytic injury was used to generate in situ thrombotic occlusion of the carotid artery in anaesthetised mice, as described (Jackson et al, Nature Medicine 2005; Schoenwaelder et al, J Biol Chem, 2007). Baseline blood flow through the carotid artery was measured using an ultrasound Doppler flow probe. The flow probe was removed, and vessel injury was delivered in the presence of stasis, through placement of a distal haemostatic clamp (Micro Serrefine Clamp, 18055-05, Fine Science Tools, Canada). Delivery of an electrical current (8 mA or 28 mA for 3 minutes) was achieved using a lesion making device (Model 53500, Ugo Basile, Comerio, VA, Italy), with the injury site flushed constantly with saline to prevent tissue dehydration. Following injury, the haemostatic clamp and electrode were removed, and the flow probe was reattached to the carotid artery to allow monitoring of blood flow for the remainder of the experimental procedure (60 minutes post injury). Stable occlusion was defined as a reduction of blood flow to 0 ml/min/100 g for 10 minutes.

Thrombolysis: Intravenous thrombolysis was commenced 15 minutes following stable thrombotic vessel occlusion, as monitored with the flow probe. rtPA was administered via jugular catheter using a Harvard Apparatus syringe pump (Cat #704504; Pump Elite 11 I/W Single Syringe Pump, NSW, Australia). (rtPA, 10 mg/kg bolus/infusion), in the absence or presence of anticoagulant. Carotid artery blood flow was assessed for 60 minutes following commencement of treatment. At the completion of intravenous infusion, the jugular catheter was removed, and the proximal (towards the heart) tie tightened to prevent blood loss. Following thrombolytic therapy, return of carotid artery blood flow was assessed using the mean blood flow recorded with LabChart software (version 7.0, ADInstruments, NSW, Australia). Recanalisation was identified by a return of blood flow, and was classified as stable, unstable, transient or none. None described persistent occlusion for the duration of monitoring with no recanalisation event/s; transient blood flow was characterised by transient recanalisation event/s followed by reocclusion; fluctuation of mean blood flow without complete vessel reocclusion was defined as unstable blood flow; stable blood flow was characterised as blood flow with no reocclusion or fluctuation in mean blood flow.

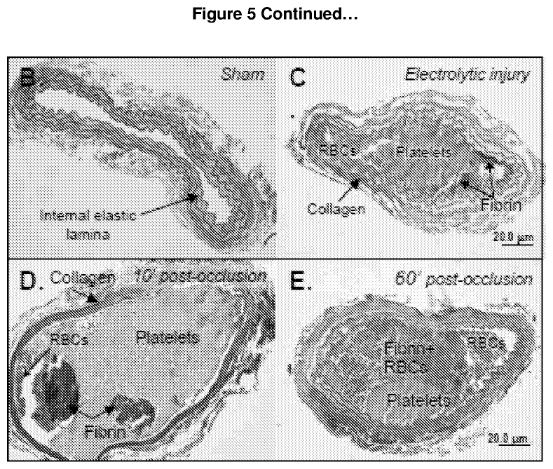

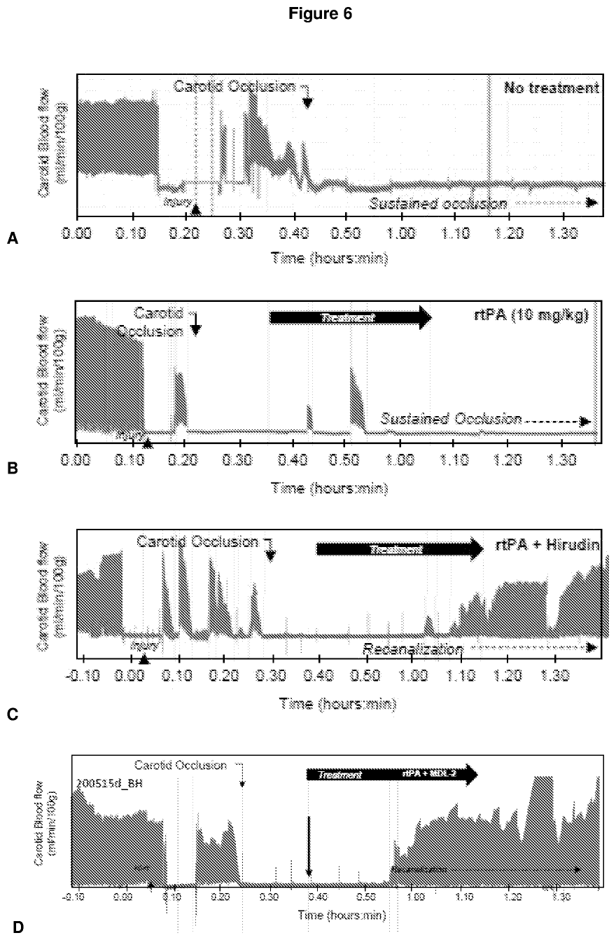

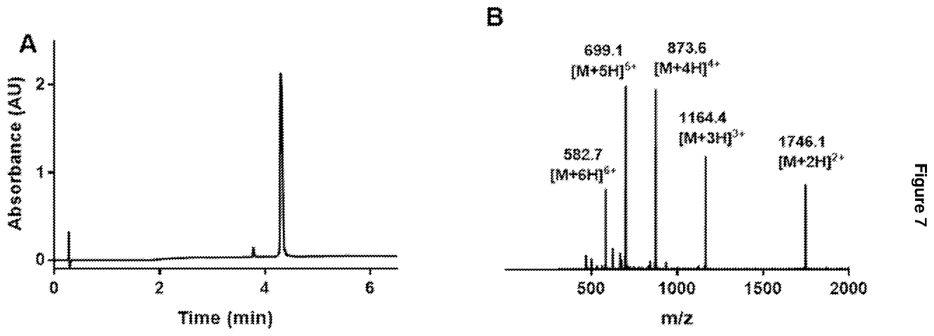

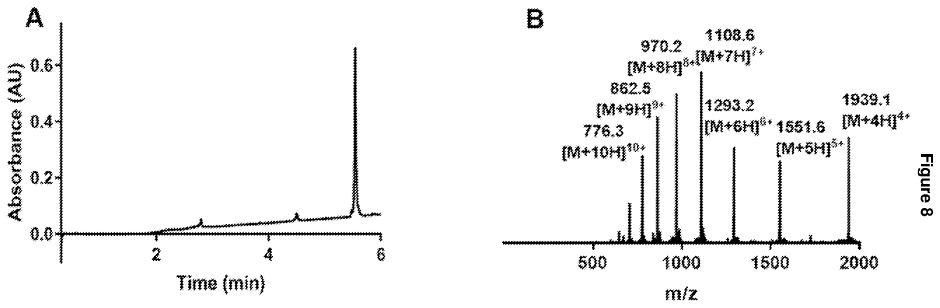

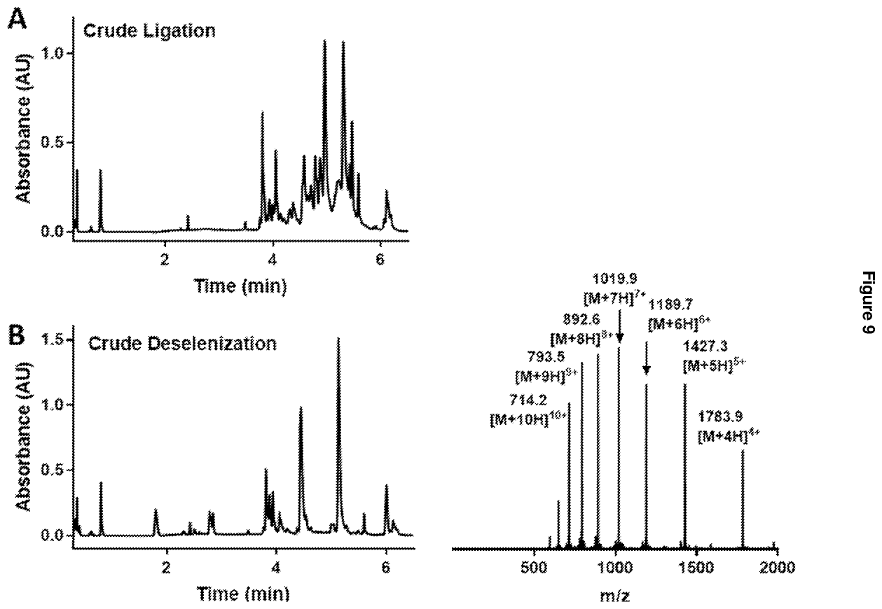

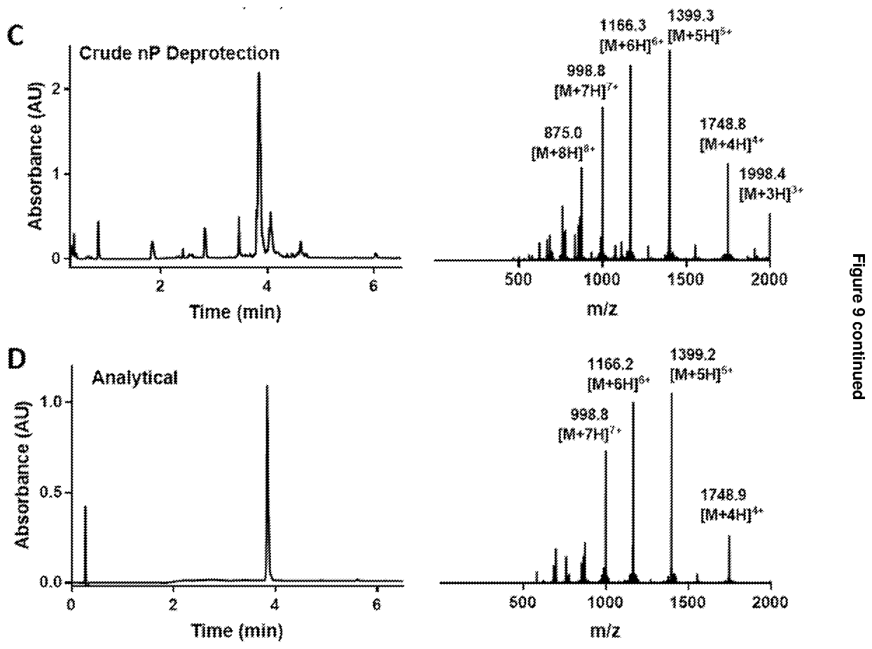

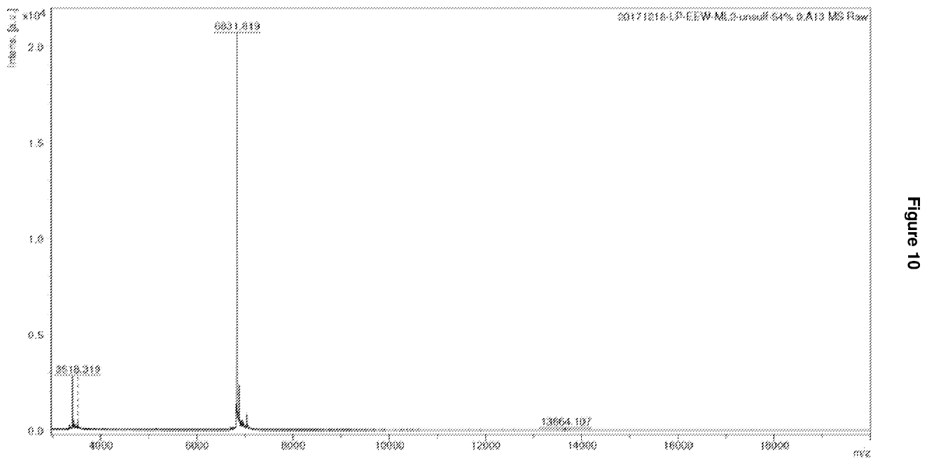

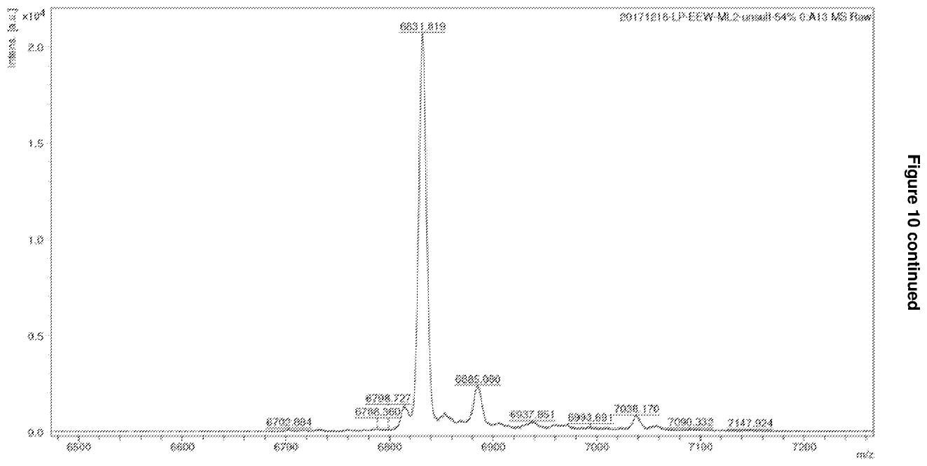

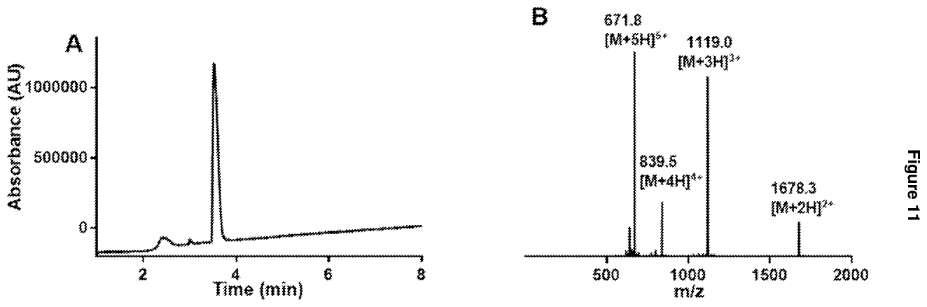

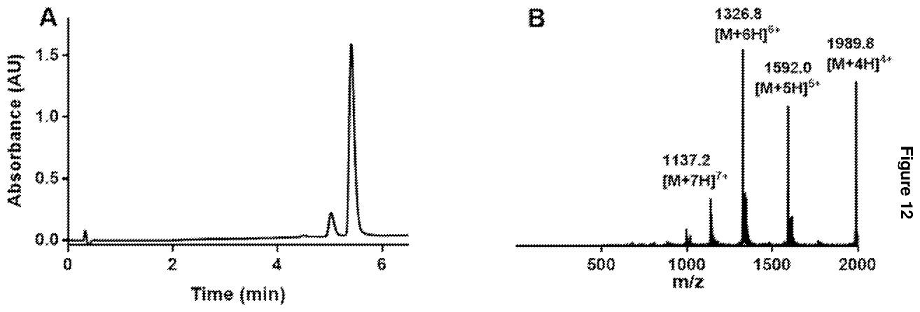

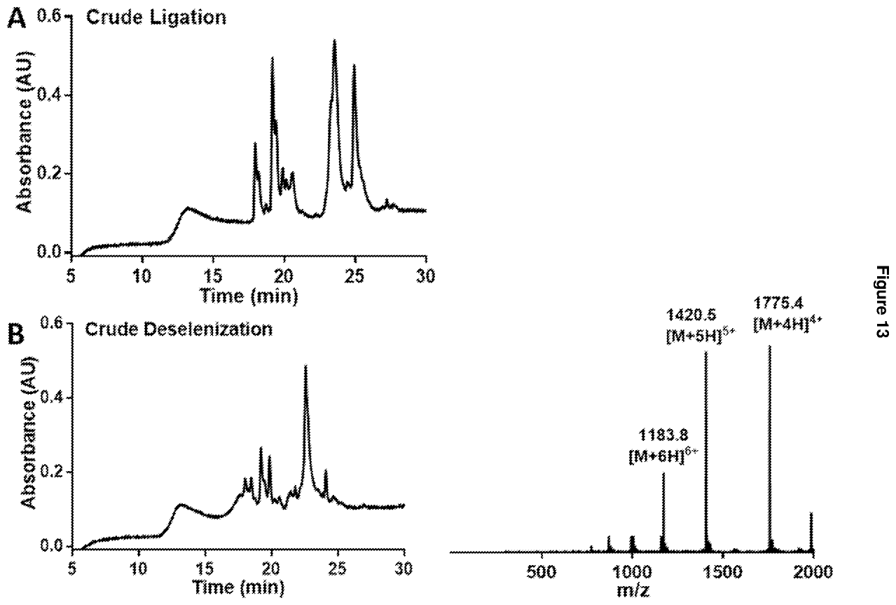

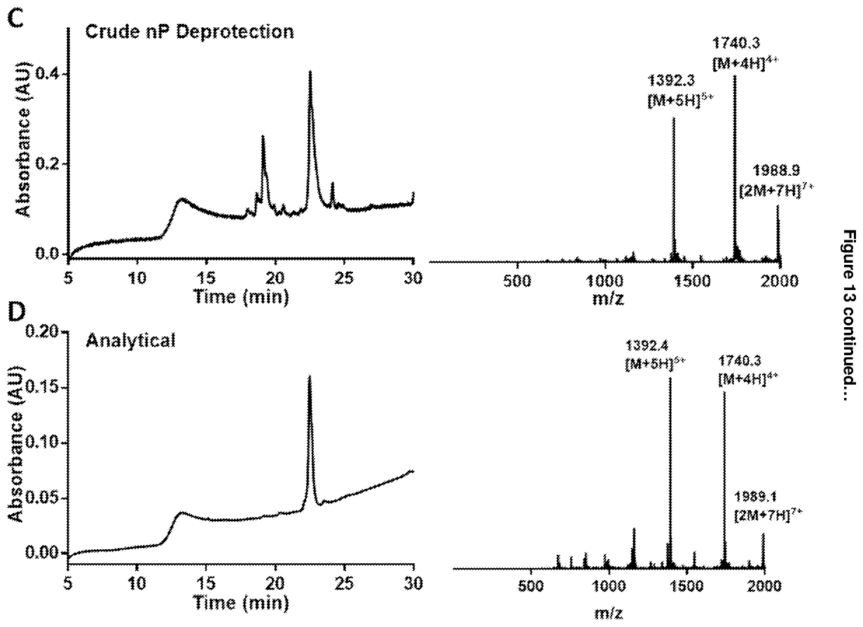

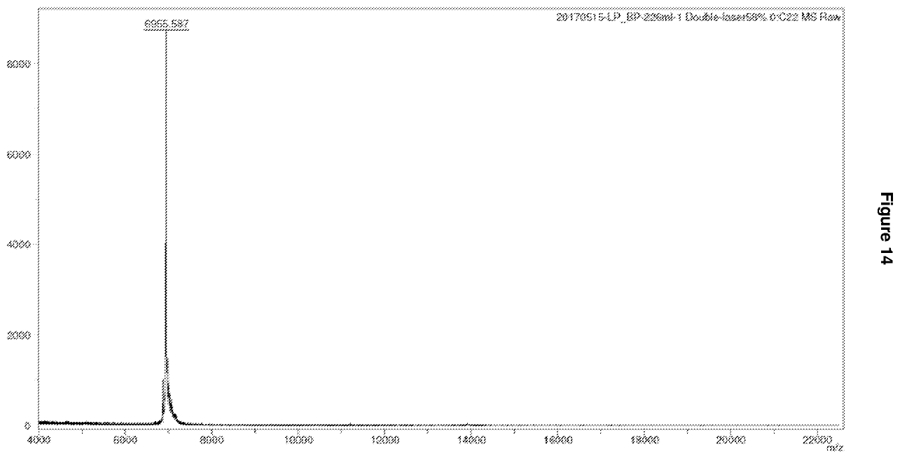

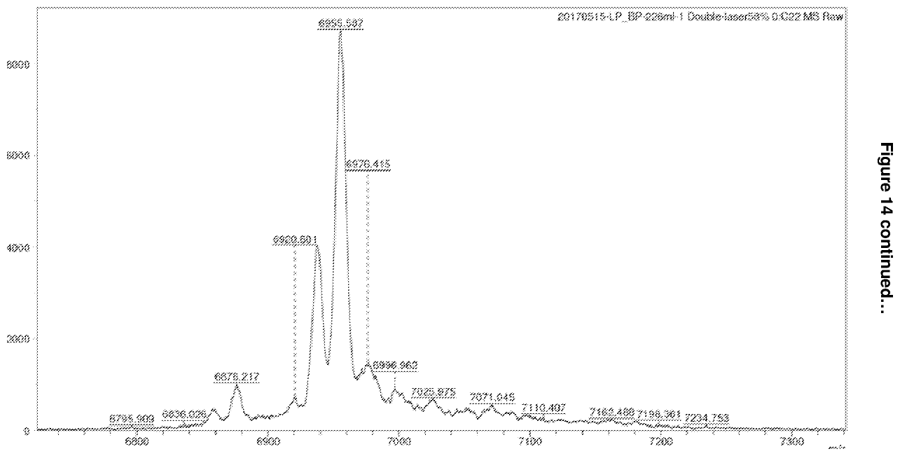

The inventors investigated the ability of a peptide of SEQ ID No:1 to recanalise an occluded blood vessel in conjunction with rtPA therapy in an experimental thrombolysis assay. A schematic of the experimental thrombolysis timeline is shown in FIG. 5A, with images of representative injuries including clot formation shown in FIG. 5B-E. FIG. 6 shows results obtained with no treatment (A), rtPA therapy alone (B), rtPA therapy in combination with hirudin (C), and rtPA therapy in combination with MDL-2 (D). These results show that the blood vessel remains occluded in the absence of treatment and with rtPA therapy alone. rtPA therapy in the presence of MDL-2 results in significant increase in blood flow compared with hirudin on a molar equivalent basis.

1. A peptide comprising, consisting essentially of, or consisting of an amino acid sequence shown in SEQ ID No: 1 or SEQ ID No: 2.

2. The peptide of claim 1, wherein at least one residue of SEQ ID No: 1 or SEQ ID No: 2 is a sulfated tyrosine residue.

3. The peptide of claim 1, wherein the tyrosine at position 32 and the tyrosine at position 35 in SEQ ID No: 1 are sulfated.

4. The peptide of claim 1, wherein the tyrosine at position 33 and the tyrosine at position 36 in SEQ ID No: 2 are sulfated.

5. The peptide of claim 1 wherein the peptide consists of an amino acid sequence shown in SEQ ID No: 1 or SEQ ID No: 2.

6. A fusion peptide of Formula 1:
A-B
wherein:
A is a peptide of claim 1;
B is a peptide having a sequence shown in one of SEQ ID No: 3 to 5.

7. The peptide of claim 1, wherein the peptide at least partially inhibits thrombin.

8. The peptide of claim 7, wherein the peptide inhibits thrombin with an inhibition constant, Ki, of less than about 500 pM, wherein the peptide comprises an amino acid sequence as shown in SEQ ID No:1 and wherein the tyrosine at position 32 and the tyrosine at position 35 in SEQ ID No: 1 are sulphated.

9. The peptide of claim 1, wherein the peptide reduces clotting time by at least 20% compared with a known thrombin inhibitor, wherein the known thrombin inhibitor induces significant blood loss, on a molar equivalent basis of the peptide and known thrombin inhibitor.

10-11. (canceled)

12. The peptide of claim 1, wherein the peptide does not induce significant blood loss.

13. The peptide of claim 1, wherein the peptide reduces blood loss by at least 10-fold compared with a known thrombin inhibitor, wherein the known thrombin inhibitor induces significant blood loss, on a molar equivalent basis of the peptide and the known thrombin inhibitor.

14. The peptide of claim 1, wherein the peptide reduces clotting time by at least 20% compared with a known thrombin inhibitor, wherein the known thrombin inhibitor induces significant blood loss, on a molar equivalent basis of the peptide and known thrombin inhibitor, wherein the known thrombin inhibitor is hirudin.

15. A pharmaceutical composition comprising a peptide of claim 1, together with a pharmaceutically effective carrier, diluent or excipient.

16. A polynucleotide comprising a nucleotide sequence encoding a peptide of claim 1.

17. A vector or expression construct comprising a polynucleotide comprising a nucleotide sequence encoding a peptide of claim 1.

18. A cell comprising a polynucleotide, vector, or expression construct encoding a peptide of claim 1.

19. The cell of claim 18 wherein the cell comprises a tyrosylprotein sulfotransferase enabling sulfation of tyrosine residues.

20. A method of preventing treating, or reducing the severity of a symptom of, a thromboembolic condition in an individual in need thereof, the method comprising, consisting essentially of or consisting of administering a therapeutically effective amount of a peptide of claim 1, the individual, thereby preventing treating, or reducing the severity of a symptom of, the thromboembolic condition in the individual.

21-23. (canceled)

24. The method of claim 20, wherein the thromboembolic condition is selected from: myocardial infarction, stroke, pulmonary embolism, deep vein thrombosis, peripheral arterial occlusion, disseminated intravascular coagulation, cardiovascular and cerebrovascular thrombosis, thrombosis associated with post-operative trauma, obesity, pregnancy, side effects of oral contraceptives, prolonged immobilization, and hypercoaguable states associated with hematologic, immunologic or rheumatological disorders, unstable angina, arteriosclerosis, a reblockage of vessels after angioplasty with a balloon catheter, or blood clotting in hemodialysis.

25. (canceled)

26. A method for inhibiting or minimising the progression of clot formation associated with inflammation caused by a viral infection in an individual comprising, consisting essentially of or consisting of administering a therapeutically effective amount of a peptide of claim 1, to an individual who is experiencing inflammation caused by a viral infection, thereby inhibiting or minimising the progression of clot formation in the individual.

27-28. (canceled)
